(19) United States
(12) Patent Application Publication
(10) Pub. No.: US 2024/0282460 A1
(43) Pub. Date: Aug. 22, 2024

(54) Method to Identify Patterns in Brain Activity

(71) Applicant: Board of Regents, The University of Texas System, Austin, TX (US)

(72) Inventors: Janani Balaji, San Antonio, TX (US); Jennifer Brethen, San Antonio, TX (US); George Britton, San Antonio, TX (US); Nicolas Grandel, San Antonio, TX (US); Chenyue Hu, San Antonio, TX (US); Zaharie Maloney, San Antonio, TX (US); Sean Tritley, San Antonio, TX (US); Byron Long, San Antonio, TX (US); Arun Mahadevan, San Antonio, TX (US); Erin Pollet, San Antonio, TX (US); Amina Ann Qutub, San Antonio, TX (US)

(73) Assignee: Board of Regents, The University of Texas System, Austin, TX (US)

(21) Appl. No.: 18/570,151

(22) PCT Filed: Jun. 14, 2022
(86) PCT No.: PCT/US2022/033490

§ 371 Date: Dec. 14, 2023

Related U.S. Application Data
(60) Provisional application No. 63/210,633, filed on Jun. 15, 2021.
(60) Provisional application No. 63/210,478, filed on Jun. 14, 2021.

(51) Int. Cl. G16H 50/50; A61B 5/00; A61B 5/0205; A61B 5/369

(52) U.S. Cl. CPC G16H 50/50; A61B 5/0205; A61B 5/369; A61B 5/4806; A61B 5/6802

(57) ABSTRACT

Methods described herein are directed to solving the problem of safely evaluating organs or tissues in a living subject and assessing and monitoring these living systems. In certain aspects the brain of a living subject is evaluated. The methods described herein integrate behavioral measurements and other non-invasive information gathering (e.g., imaging, EEG, etc.) or minimally invasive information gathering (e.g., biological fluid sampling) with cellular imaging, and biomimetic models to safely evaluate a subject. In vitro models are established that can be manipulated and monitored on the cellular level.

### RELATED APPLICATIONS

This application claims priority to U.S. Provisional Application Ser. No. 63/210,478 filed Jun. 14, 2021 and U.S. Provisional Application Ser. No. 63/210,633 filed Jun. 15, 2021, both of which are incorporated by reference in their entirety.

### STATEMENT REGARDING FEDERALLY FUNDED RESEARCH

This invention was made with government support under 1533708 and 182560 awarded by the National Science Foundation. The government has certain rights in the invention.

### BACKGROUND

Certain embodiments are directed generally to the field of living systems engineering, artificial intelligence, and neurovascular biology.

Understanding how the brain rejuvenates on a daily basis has the potential to transform what is known of human physiology. In certain aspects, methods are presented to evaluate the process of brain cells repair by monitoring the changing of cell fate and function during rest. Humans spend a quarter to a third of our life sleeping, and sleep is known to be essential for human health (9). Cognitive processing (10), memory formation (11) and immune response (12) all rely on sleep. Even one night of sleep deprivation in healthy adults shows adverse effects, including higher Aβ accumulation (13). Disrupted sleep and sleep duration has also been correlated to, and even predictive of, cognitive decline (14). Studies have also shown protective changes in the brain's activity and blood flow during sleep (15). However, despite advances at the macroscale, very little is known about what happens at the cellular level to contribute to cognitive health. Whether, when, or how brain cells change shape, function and communication are uncharted research areas due to our inability to safely access the human brain in living subjects, or in animals over long timespans in natural settings.

There remains a need for additional methods of evaluating a system at the cellular level.

### SUMMARY

The intellectual merit of this work lies in its ability to use modeling to address a previously intractable problem, enabling observations of human cells and behaviors not previously possible. The experimental design also provides an uncommon testbed where brain changes for subjects is recorded and whose living “brain models” are evaluated in the lab.

Methods described herein are directed to solving the problem of safely evaluating organs or tissues in a living subject and assessing and monitoring these living systems. In certain aspects the brain of a living subject is evaluated. The methods described herein integrate behavioral measurements and other non-invasive information gathering (e.g., imaging, EEG, etc.) or minimally invasive information gathering (e.g., biological fluid sampling) with cellular imaging, and biomimetic models to safely evaluate a subject. In vitro models are established that can be manipulated and monitored on the cellular level. Living subjects are monitored and various data gathered through non-invasive or minimally invasive measurements. The in vitro information is then used to interpret and analyze the non-invasive information providing an evaluation or assessment of the target organ or tissue in a living subject. In certain aspects the system level information is translated to the cellular level observations obtained in vitro.

Certain embodiments are directed to methods of evaluating a living system comprising: (a) measuring in vivo physiologic, behavioral, or physiologic and behavioral characteristics of a living subject to obtain non-invasive data; (b) establishing an in vitro cell model of a cellular network, exposing the in vitro cell model to a condition(s) to model a cellular environment in the living subject, and measuring cellular changes to obtain in vitro model data; (c) transforming the non-invasive data to functional graphs; (d) transforming the in vitro model data to topological, functional, or topological and functional graphs; and (e) integrating the non-invasive graph(s) and the in vitro model graph(s) using a neural network or a combination of neural networks. A neural network has two primary functions. One is a “processing” function to obtain an output from inputs, and the other is a “learning function” to set a relationship between an input and an output of a whole neural network to a desired relationship. In certain aspects, the transforming the non-invasive data to topological and functional graphs utilizes cytoNet software or an equivalent. In certain aspects, transforming the in vitro model data to topological and functional graphs utilizes cytoNet software or the equivalent. The non-invasive data can be one or more of non-invasive imaging, biomarker analysis, or bio-electrical patterns. Physiologic information can be obtained by use of electrocardiogram (ECG), photoplethysmography (PPG), electroencephalogram (EEG), galvanic skin response (GSR), electrogastrography (EGG), electromyogram (EMG), electrooculogram (EOG), polysomnogram (PSG), temperature, etc. In certain aspects, the bio-electrical patterns can be electroencephalograms (EEG) or polysomnograms (PSG). In certain aspects the non-invasive imaging is a retinal scan(s). The non-invasive imaging can be biomarker analysis of a blood sample or other biological sample such cerebrospinal fluid, saliva, lymph, urine, or stool sample. In certain aspects the neural network is a long short-term memory network (CNN-LSTM) or other neural network or deep learning algorithm or system.

Certain embodiments are directed to methods or processed for identifying unique sleep patterns or sleep signatures predictive of cognitive performance change in response to an activity, for example exercise, using data from sensor devices or wearables.

Other embodiments are directed to molecular (epigenetic) and cellular (neuronal) biomarkers of sleep quality.

Certain embodiments are directed to experimental, human cell-based brain models of regions of the brain regulating sleep, mood, and circadian rhythmicity (quantitative, living models of the brain's suprachiasmatic nucleus).

Other embodiments are directed to methods and/or models to predict the brain's response to exercise and sleep—and associated chemical regulators—from molecular to behavior.

A “sleep signature” is used herein in the broadest sense and is used to refer to a pattern of sleep stages common between two or more sleep stage sequences. Sequence motifs can be readily identified by a variety of pattern discovery algorithms. Certain embodiments are directed to methods for defining a sleep signature for a subject comprising: (a) plotting frequencies of sleep stages of a subject over a period of time, wherein the sleep stages are light sleep (L), deep sleep (D), rapid eye movement (REM) sleep, and wake (W); (b) identifying sleep stage motifs in the sleep stage plot. In certain aspects the sleep stages are determined using a wearable device, e.g., FITBIT™ or the equivalent. The sleep stage motifs can be identified by scanning a window of sleep stage sequence by comparing a scan window to a position frequency matrix and assessing a probability of the sequence using a position probability matrix and determining the probability of the motif. The identification of sleep signatures or motifs starts with the use of a database of related sleep stage sequences and the identification of sequence motifs that are shared by individual members. Various computer programs can be used for identifying sequence motifs. One or more sequence motifs identified are aligned to each other, and subdivided into separate datasets, each dataset being characterized by sharing a predetermined combination of parameters characteristic of one or more of the aligned sequence motifs. Such parameter can, for example, be the duration or timing of the sleep stage, the subfamily in which a particular sequence motif belongs, the population from which the sequence derives, etc. The datasets characterized by a given combination of two or more parameters are then analyzed by position for frequency usage to identify key usage in individual stretches of sleep stages within the datasets. Steps in the process of frequent sequence analysis of sensor data include, for example: (i) binning time-course data into intervals of interest (e.g., 30 sec epochs to quarters of nights); (ii) representing sleep stages as letters and/or letters and number (the latter, for duration). As an example, substituting nucleic acids (A,T,C,G) with the stages of sleep recorded by the Fitbit device (deep, REM, light, wake) to identify frequent sequences (e.g., deep-light-REM=dlr or d10-12-r15 based on the duration and sequence of these sleep stages) by sequence analysis; (iii) identifying the frequency and distribution in which certain letters and letter-number pairs, and combinations of letters and letter-number pairs occur across a whole population of individuals and across specific demographics (e.g., age, gender) within single nights and/or across many nights; (iv) identifying sequence motifs (letters, letter-number pairs and/or strings of letter and letter-number pairs) that occur more frequently than average during specific time intervals for an individual and across individuals; (v) Ranking these frequent sleep motifs by frequency. In a first analysis of sensor-based tracking, the inventors ranked the sleep motifs most representative of specific demographics, including age; (vi) Training predictive models on the sleep stage and motif patterns for individuals and populations; (vii) Predicting (e.g., by random forest and/or deep neural networks) future sequences of sleep motifs based on behavior, gender, age, adherence to a new drug therapy, disease-status or other demographics.

Certain embodiments are directed to methods of identifying sleep motifs comprising: (i) generating a sleep signature by obtaining EEG, (ii) measuring and analyzing heartrate data to characterize deviations in circadian rhythm, and/or (iii) analyzing sleep data to find motifs in the data.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. General schematic of wake/sleep cycle. This work characterizes cell dynamics in the human brain and test the hypothesis that brain cells repair by changing their fate and function during rest. Left circle: confocal image from a neurogenesis assays (MAP2; Ki67; DAPI; nestin; 20×)

FIG. 2. Brain changes studied non-invasively in volunteers whose living “brain models” are designed, studied and validated in the lab. The schematic summarizes experiments and measurements to be performed. Schematic produced using bioRender with microscopy images from neurogenesis assays and an example of a volunteer's EEG recording from a Dreem device.

FIGS. 3A-3D. Sleep signature identification from wearable device recordings. (A) A wearable device (Fitbit™ Charge 3) vs. polysomnography (PSG) comparisons across a sleep hypnogram. (B) Logo plots for representative young (top) and older (bottom) volunteers highlighting identified frequent sequences in sleep. Height of the lettering represents frequency. (C) Summary of the motif-finding approach. (D) Ranking of the 10 most frequent sleep motifs for younger (left) and older (right) volunteers from 2+ months of nightly data.

FIG. 4. Example of the results showing differentially changing chromatin markers from the blood of volunteers (RNA55P528, FRG2C, BCL7B, FAM230C); identification of changes in cognitive test performance (positive or negative) based on the predictions of the acrophase of rapid eye movement sleep; and a display of the test scores changes for nine metrics for one volunteer across the exercise intervention.

FIG. 5. Steps in brain model design.

FIGS. 6A-6E. An example of the methods to design and characterize brain models. Step 1: (A) Day 7 iPSC NCRM-5 neural network (1). (B) Detection of dendrite length and branching during neurodifferentiation (2). (C) Graph creation across biological scales and imaging modalities (4,5): cell morphology mapped to graph properties (fixed cells); (D,E) soma (nodes) and dendrites (edges) tracked live.

FIG. 7. Step 2: Physiologically active iPSC model. TuJ1, neurons. Fluo-4, Ca2+ signaling dye. DAPI, nuclei. VGAT, inhibitory synapses. NCRM-5 shown. 20×. Schematic of the structure-functional graphs. Coordinated topological-functional network development shown for differentiating ReNCells. Step 3: Stress and sleep regulator analog assays.

FIG. 8. AI-graph hybrid method predicts brain cell repair during sleep Same color=measurements taken in the same system simultaneously. Same symbol=same form of data (electrical, morphological), including features from cytoNet and features from the sleep signature analysis.

FIG. 9. Rule, imaging and agent-based modeling approach to map from low resolution images to high resolution predictions of cell behaviors.

FIG. 10. cytoNet characterizes a neurodevelopmental disorder (A) iPSC neural networks from a child with Smith-Lemli Optiz Syndrome (SLOS, CWI 4F2) are compared to a control line (NCRM-5) (left) at day 3, and after exposure to WNT inhibitor CHIR1. (B) Astrocytes from wild-type and a SLOS model DHCR7 mutant and their Ca2+ recordings after glutamate stimulation, color-coded by astrocyte region. SLOS cells: K. Francis, Sanford Research (3).

FIGS. 11A-11C. cytoNet workflow. (a) The cytoNet pipeline begins with masks and optionally microscope images, which can be static immunofluorescence images or calcium image sequences. (b) Spatial proximity is determined either by measuring shared pixels between cell pairs—type I networks, or by comparing the distance between cell centroids to a threshold distance—type II networks (right panel). Functional networks are estimated from correlations in calcium time series data. (c) Cell community descriptors provide information on local neighborhood characteristics of individual cells, like degree and centrality measures, and global neighborhood characteristics like modularity and path lengths.

FIGS. 12A-12G. Dynamics of spatial and functional topology in developing neural progenitor cells (NPCs). (a) Spatial NPC networks at day 1, 3 and 5 of differentiation, overlaid on immunofluorescence images of nuclei stained with Hoescht dye; segmented cells are outlined, and spatial proximity edges are shown. (b) Network efficiency of spatial NPC networks peaks at day 3; notches show mean and standard deviation; *p<0.005 from two-sample t-test. (c) Functional networks obtained through calcium imaging with Fluo—4 in developing NPC networks at days 1, 3 and 5. Correlations between calcium traces from individual cells are shown as a network plot overlaid on the maximum intensity image from calcium image sequences; scale bar=50 μm for panels a and c. (d) Fraction of active cells in the network; *p<0.005 from two-sample t-test. Active cells are defined as cells whose normalized fluorescence traces have three or more calcium transients. (e) Frames from time-lapse movies of differentiating NPCs transfected with FUCCI cell cycle reporters. Borders of mCherry+ nuclei (G1) are outlined, Venus+ nuclei (S/G2/M) are outlined, and mCherry−/Venus− nuclei (quiescent) are outlined, spatial edges are overlaid; scale bar=50 μm. (f) Neighborhood similarity score for low-density culture across time. (g) Neighborhood similarity score across time for medium-density culture. FIGS. 2a-d adapted from reference (2).

FIGS. 13A-13B. Dynamics of Coupled Functional and Spatial Analysis In Vivo. cytoNet captures relationships between spatial proximity of neurons and functional features of multicellular modules in vivo. (a) Cells classified according to the time required to first reach their maximum ΔF/F0 values from 20% of that value (ramp-up) and the time required to return to 20% (ramp-down). Edges connect similarly classified cells that are within 10 cell diameters of each other. All cells reached their peak values at 20 seconds except for those circled which reached their peak values at 25 seconds. (b) Calcium time series (ΔF/F0) plotted for 6 categories of cells with unique combinations of ramp-up and ramp-down times. The blue braces indicate a cell's ramp-up and ramp-down. Each inset image is a spatial pattern of cells with the same ramp-up and ramp-down times. RU=ramp-up time; RD=ramp-down.

FIGS. 14A-14H. Influence of local neighborhood density on primary human endothelial cell (HUVEC) morphology. (a) Distribution of cell circularity values grouped under different levels of closeness centrality; sample size, n=786 cells (group 1; cₙ<0.025), 741 cells (group 2; 0.025<cₙ<0.05) and 782 cells (group 3; cₙ>0.05); Cohen's d effect size: groups (1, 2)=0.34, groups (1, 3)=0.62 (b) Sample immunofluorescence image with graph representation overlaid; scale bar=50 μm. (c) Heatmap depicting closeness centrality of each cell, with circularity values overlaid in text. (d) Representative cells from cluster analysis, highlighted in magenta. (e) Cell size, closeness centrality and circularity distribution plots for each cluster. (f) Bar plot of variance explained by growth factor treatment and local network metrics. (g) Box plot of cell size as a function of growth factor treatment. (h) Box plot of mean actin intensity as a function of growth factor treatment. Legends and axes in (f-h) contain information on treatment (BDNF, VEGF), concentration (50 ng/ml, 100 ng/ml) and time of treatment (6 hours and 12 hours). Cohen's d effect size for (f-h) is shown in Supplementary Table 2.

FIG. 15A-15H. Spatial Analysis of the Pericapillary Niche in Adipose Tissue. Example confocal images of wild type (a) and knock out (b) adipose tissue and the corresponding output graph for the wild type image (e). Red=lectin (capillaries). Green=Bodipy (adipocytes). Yellow: integrin a7 positive cells. Violin plots of cell properties comparing wild-type and knockout (c, d, f-h). Distances are measured between the closest border pixels of pairs of objects. FIG. 6f is adapted from reference (1). Error bars are mean+/−standard deviation. p-values were computed using the Wilcoxon rank sum test (***: p≤0.001).

FIG. 16A-16F. Image segmentation of HUVEC immunofluorescence images. (a) Original grayscale image. (b) Image after adaptive histogram equalization and Gaussian filtering. (c) Binary image obtained using Otsu's threshold, with small objects removed. (d) Complement of filtered image in (b). (e) Watershed basins obtained through imposing minimum of images in (d) and the marker image (obtained by combining the binary image in (c) and the image obtained through binarization of microtubules and nuclei). (f) Final cell borders.

FIG. 17A-17D. Image processing steps for FUCCI-ReN nucleus images. (a) Fluorescence image from H2B—Cerulean channel marking all nuclei. (b) Binary mask obtained through adaptive thresholding. (c) Image obtained through imposing minimum of distance transform of binary image in (b) and local minima. This image serves as seeds for the watershed algorithm. (d) Final mask obtained after watershed transform

FIG. 18. Correlation heatmap of local network metrics and morphology metrics for immunofluorescence HUVEC images. All morphology and local network metrics (Supplementary Table 1, Supplementary Table 2) were combined into a single matrix. The cluster dendrogram was obtained through hierarchical clustering of the covariance matrix using Pearson's correlation as the similarity metric.

FIG. 19A-19C. Designing brain models by cytoNet Pattern analysis, step 1.

FIG. 20. Designing brain models by cytoNet Pattern analysis, step 2.

FIG. 21. Designing brain models by cytoNet Pattern analysis, step 3.

FIG. 22. Designing brain models by cytoNet Pattern analysis, step 4.

FIG. 23. A schematic of one example of a cell differentiation protocol.

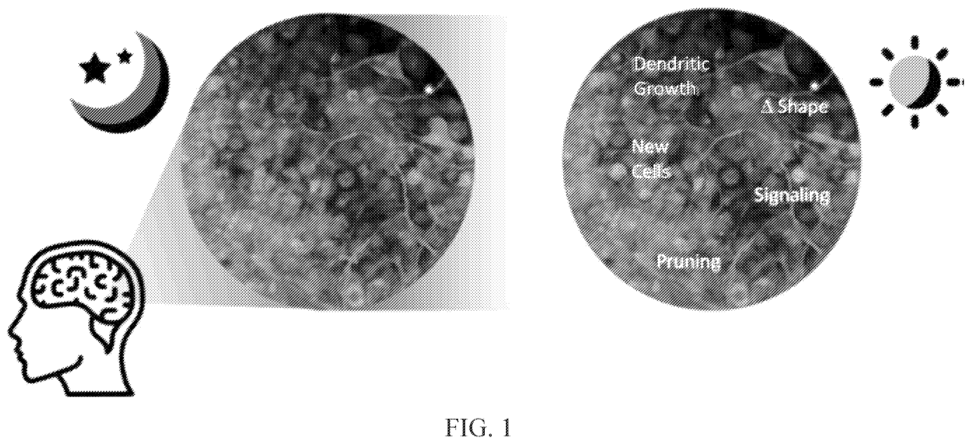

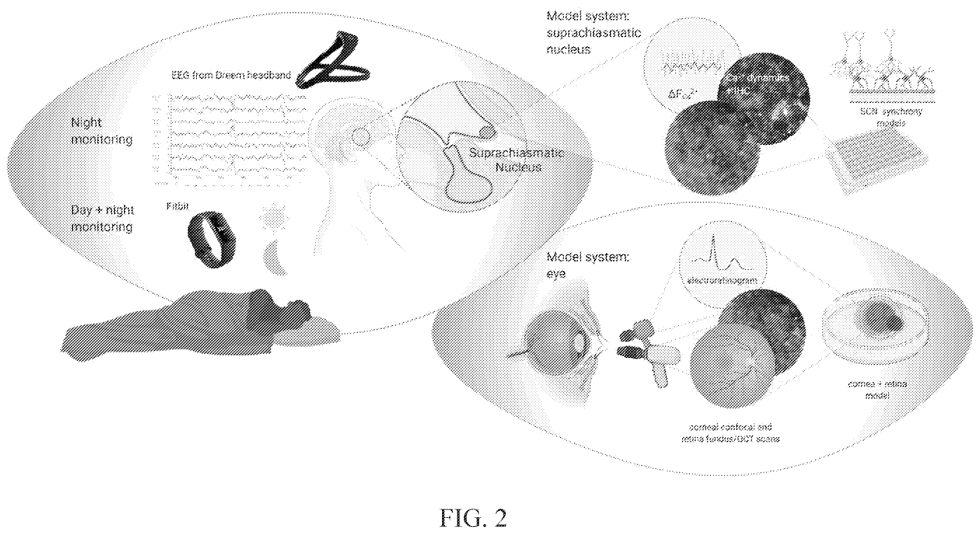

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

The intellectual merit of this work lies in its ability to use modeling to address the previously intractable problem of observing cellular characteristics and behaviors in a living subject, enabling observations of human cells and behaviors not previously possible. Whether, when, or how brain cells change shape, function and communication are uncharted research areas due to our inability to safely access the human brain in living subjects, or in animals over long timespans in natural settings. The first proof-of-principle in humans includes the linking of daily behaviors to structural and functional changes in brain cells. Using an integration of sensor-based behavioral measurements, cellular resolution imaging in humans, and biomimetic models, a system is developed that tests (1) cells in an adult brain are not committed to a single role or cell type, and (2) change in function (and even change in cell size and structure) is characteristic of health and necessary for repair from daily stresses. The study design provides an uncommon testbed. Brain changes for volunteers are recorded whose living “brain models” are designed in the lab.

The impact of the methods described stems from (1) the introduction of three innovative techniques that can be broadly applied to study cellular changes in humans; (2) new biomimetic models that recapitulate key parts of the brain that regulate sleep; (3) integrative algorithms that enable predictions of cell changes from non-invasive recordings; and (4) a first proof-of-principle of the behavior-to-cells prototype to study how the human brain changes as a function of daily activity.

The ability to “see” into the human brain and identify how brain cells change as a function of daily activity can transform what we know about physiology. The methods span domains (sensor-based tracking, living engineered systems, artificial intelligence, neuroscience), scales (behaviors to cells), and systems (human, biomimetic models, computer models). The complexity of the problem demands a convergence of new technologies and conceptional innovations.

Studying cell dynamics in healthy, living humans is an uncharted area. What human brain cells are doing as a function of rest has only been studied circumstantially, at a macroscale or in other organisms. In the human brain during sleep, cerebrospinal fluid clears toxins and activates electrical waves (15), however, cellular phenotypic and molecular changes remain unknown. The prevailing paradigm views adult brain cells as largely static (42). However, hints from other organisms suggest otherwise. Reactivation of neural stem cells was recently observed in the fruit fly (43,44); prior work identified oncogenic factors that induce dedifferentiation of neurons in vivo (45); and in vivo genetic reprogramming of neurons shows the feasibility of inducing cell fate changes in the living brain (46,47). Moreover, recent work in animal models (rat, mice, zebrafish) during sleep support our hypothesis of daily cellular repair. These studies demonstrated microglia (48), glia (49) and neurons (48,50) change shape and move during sleep. In the brains of fruit flies, ionic flux and neuron synaptic morphology has also been observed to change as a function of circadian clock genes and control sleep quality (51). Despite these exciting advances in vivo, translating them to the human brain remains challenging. The hurdle to studying cell repair in humans is multi-fold: (1) resolution limits of non-invasive, live imaging (52); (2) the uniqueness of the human brain; and (3) lack of experimental frameworks to measure how human behavior affects cellular changes.

Each of the studies provides new technical advances and biological insight. From 200 healthy volunteers whose sleep has been tracked daily (6-36 months), a first test is whether biomarkers of DNA repair and neurogenesis change in the blood as a function of sleep. In a second test, biomimetic models of the cornea, vascularized retina and suprachiasmatic nucleus for individuals with extremes of blood-borne DNA repair and sleep quality will be developed. These models enable us to study regions of the human brain regulating sleep, where experimental conditions can be designed to mimic conditions that people may experience (e.g., oxidative stress, circadian rhythm disruption). Stress conditions will be studied in these brain models and compared to measurements obtained by non-invasive imaging and electrical recordings in the human brain. In a third study, AI-graph hybrid models are developed that train on coupled electrical, biochemical and morphological data in humans and in vitro. These models will be used to predict how brain cells change as a function of rest. Together, the studies and modeling provide the foundation for a new field of research: the study of human cell plasticity in vivo.

The methods described herein can elucidate what is happening at the cellular level, for example elucidating the cellular activity in our brains when we rest. Methods and models developed can be used broadly to test additional theories of brain physiology and guide new behavioral interventions or therapies to improve brain repair. Technical products of this work include the ability to predict brain cell changes from non-invasive EEG, electroretinogram and activity recordings. Technical advances can transform future diagnosis options and identify new therapeutic candidates for the 1 Billion people worldwide suffering from neurological disorders. Across application areas, the behavior-to-cells experimental platform that links non-invasive measurements to cell phenomena used as a prototype for individualized, quantitative multiscale studies of human physiology. Broader impacts also include new open-source AI algorithms and stem cell differentiation protocols for the research community. Brain health research workshops, biomedical data workshops, and dissemination of the work through online interactive data visualizations will directly engage the public and student trainees in fostering brain health and learning interdisciplinary technologies in bioengineering.

Three innovative engineering technologies will overcome these hurdles: (A) Unique experimental testbed. Daily activity and brain changes are recorded for volunteers whose living “brain models” are studied in the lab. In addition to elucidating the structure-function of the human system, these models can lay the groundwork for regenerative medicine applications that incorporate living tissue constructs. (B) AI-graph hybrid models to predict structure from activity. To link the structure-function relationships observed in brain cells, a graph-based framework (1,4) is developed. Here, the graph paradigm is integrated with a convolution neural network to enable us to improve the accuracy of both approaches. Once validated, the predictive models provide a platform to test numerous conditions that may change the ability of the brain to repair. (C) Predictions of brain changes across scales (behavior to cells). The methods to predict cell changes from non-invasive recordings would enable a way to ‘see’ into the living human brain with minimal risks. Additionally, the models link systems-to-cell level data.

### I. CYTONET

cytoNet provides an online tool to rapidly characterize relationships between objects within images and video frames. To study complex tissue, cell, and subcellular topologies, cytoNet integrates vision science with the mathematical technique of graph theory. This allows the method to simultaneously identify environmental effects on single cells and on network topology. cytoNet has versatile use across neuroscience, stem cell biology, and regenerative medicine. cytoNet applications described include, but are not limited to: (1) characterizing how sensing pain alters neural circuit activity, (2) quantifying how vascular cells respond to neurotrophic stimuli overexpressed in the brain after injury or exercise, (3) delineating features of fat tissue that may confer resistance to obesity and (4) uncovering structure-function relationships of human stem cells as they transform into neurons.

Discoveries in biology increasing rely on images and their analysis (106). Advances in microscopy and accompanying image analysis software have enabled quantitative description of single-cell features including morphology, gene expression, and protein expression at unprecedented levels of detail (107-110). There has also been a growing appreciation of spatial and density-dependent effects on cell phenotype. Various types of cell-cell interactions including juxtacrine and paracrine signaling are an integral part of biological processes that affect the behavior of individual cells. In response to this realization, many research groups have developed in situ profiling techniques to extract highly multiplexed single-cell data while preserving the spatial characteristics of biological samples (107, 111-115).

Need for a user-friendly tool to test biological hypotheses that depend on spatial information. The increasing prevalence of spatially detailed imaging datasets has led to the proliferation of spatial analysis pipelines for biological research (Table 1). While these methods have enabled principled exploration of spatial hypotheses, the majority of the pipelines (with a few exceptions) have been developed for spatial molecular expression data obtained through methods such as mass cytometry, specialized high-resolution imaging, and/or scRNA-seq, with inherent idiosyncrasies. Others have focused on histology and samples obtained for medical applications. As a result, these techniques are not applicable to many standard imaging datasets obtained through routine biological experiments. Further, many pipelines require the user to be familiar with programming and involve the use of customized scripts. All of these limitations mean the most advanced spatial analysis platforms are not commonly employed by biologists. Instead, the spatial analysis platforms are largely used by a subset of labs heavily invested in computational analysis, by core users of specialized microscopy, or by imaging experts themselves. There remains a need for a generalizable, easy-to-use analysis method to test spatial hypotheses applicable to a wide variety of biological imaging data.

TABLE 1
Software tools for spatial analysis

| Software | Platform | Input | Output | Reference b |
|---|---|---|---|---|
| histoCAT | MATLAB, | Imaging mass | User-guided cell neighborhood for | (50) |
| | standalone | cytometry | selected cells, enrichments/depletion of | |
| | program | | cell-cell interactions based on comparison | |
| | | | to spatially randomized data | |
| Pelkmans | Module | Cell cultures | Local cell density, population | (34, 49, |
| lab | compatible with | | size, cell islet edges | 51, 52) |
| | CellProfiler | | | |
| Cell-graph | Standalone | H&E stained tissue | Multiple graph metrics, e.g, clustering | (15) |
| | tool | samples | coefficient, network diameter | |
| PySpacell | Python | Cell cultures | Statistical tests of magnitude and | (53) |
| | | | scale of spatial effects | |
| SpatialDE | Python | Spatial | Statistical tests of genes with spatial | (54) |
| | | transcriptomics | variation, spatial gene-clustering | |
| | | datasets | | |
| trendsceek | R | Spatial | Statistical tests of genes | (55) |
| | | transcriptomics | with spatial variation | |
| | | datasets | | |
| cytoMAP | MATLAB | Histo-cytometry | Multi-scale characterization | (56) |
| | | data | of tissue structure | |
| MuSIC | Cytoscope | Immunofluorescence | Intracellular protein | (57) |
| | | and affinity | positions and distances | |
| | | purification mass | | |
| | | spectrometry data | | |

Need to capture time-dependency in structure-function relationships. In addition to spatial and morphological characteristics, time-dependent properties of cell function also define phenotype. The behavior of cell groups often includes coordinated responses of subgroups (such as in brain and heart tissue) that require intricate communication, and the role a cell plays in this communication is part of its phenotype. Live reporters and activity-based dyes can provide insight into this time-dependent cell communication. As an example, calcium imaging is a versatile technique to investigate the dynamics of cell signaling, particularly in neural and cardiac tissue. While there exist many automated tools for calcium signal analysis (Table 2), combined analysis of spatial and functional topology has the potential to reveal fundamental insight into the nature of structure-function coupling in biological systems.

TABLE 2
Software tools for calcium signal analysis.

| Software | Platform | Input | Output | Reference b |
|---|---|---|---|---|
| unnamed | MATLAB | Images | Segmentation, signal extraction, stimulus response analysis, | (58) |
| | | | assembly detection, network dynamics analysis | |
| CalmAn | Python | Images | Motion correction, source extraction, deconvolution, registration | (59) |
| EZcalcium | MATLAB | Images | Motion correction, segmentation, signal extraction, deconvolution | (60) |
| NA³ | ImageJ, R | Images | Total activity value, & variance area | (61) |
| CAVE | MATLAB | Images | Motion correction, ΔF/F calculation, cell detection, | (62) |
| | | | calcium trace analysis | |
| CaSiAn | Java | Signal | Peak and nadir detection, interspike interval and average | (63) |
| | | data | period regression, signal correlation | |
| STMA | Python | Images | Motion correction, segmentation, signal extraction, | (64) |
| | | | ROI registration | |
| Suite2p | MATLAB, | Images | Image registration, ROI detection, cell determination, | (65) |
| | Python | | activity and neuropil extraction, spike deconvolution | |
| CNMF-E | MATLAB | Images | Contour detection, signal extraction | (66) |
| ABLE | MATLAB | Images | Contour detection, neuropil correction, signal extraction | (67) |
| SCALPEL | R | Images | Segmentation, signal extraction | (68) |
| MINIPIPE | MATLAB | Images | Motion correction, segmentation, signal extraction, | (69) |
| | | | deconvolution | |
| SamuROI | Python | Images | Image stabilization, event detection | (70) |

Network science framework. A single modeling framework to represent multiple descriptors of cell community is necessary to provide continuity across spatial and temporal scales. Network science offers this modeling framework. Network science seeks to understand complex systems by representing individual functional units of the system as nodes and their relationships as edges. This abstract representation is then used to describe, explain, or predict the behavior of the system (116). Network models have been tremendously useful in studying complex biological systems, most prominently in neuroscience (116, 117). The inventors posit that network models provide a flexible, intuitive method to model spatial and functional cell community relationships. Among existing image-based analyses that employ network science, the cell-graph technique (118) has been employed to great effect in analyzing structure-function relationships in fixed tissue sections. The inventors have applied network analysis to fixed samples also enabled rapid classification of cell phenotypes (119, 120). However, the scope of network models in describing cell community structure and dynamics has yet to be fully explored.

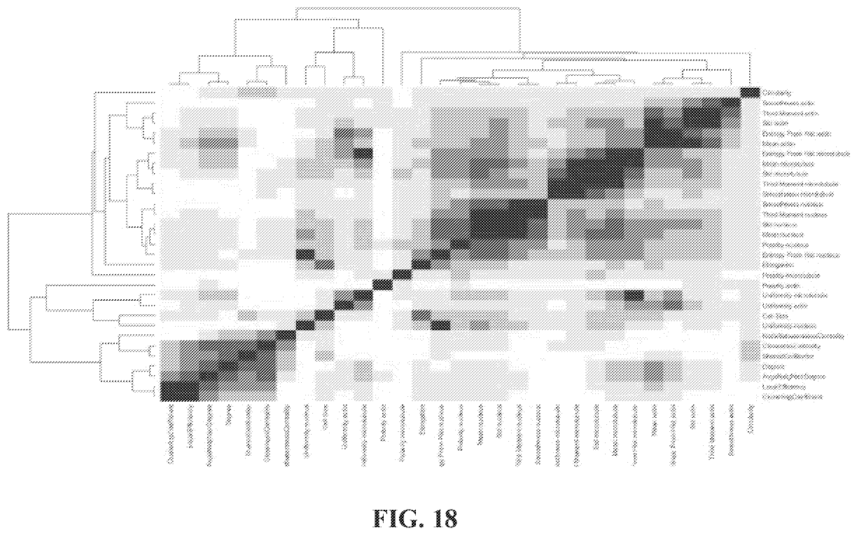

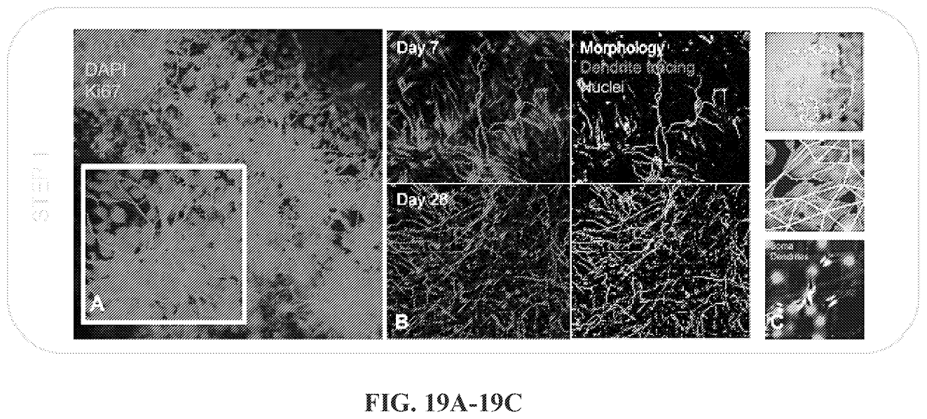

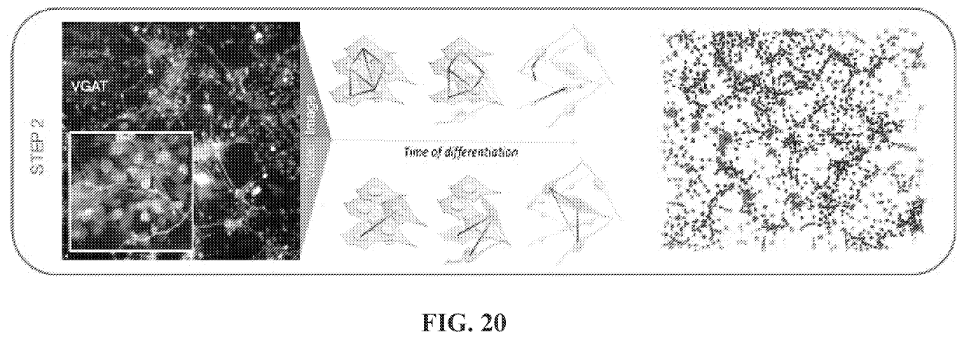

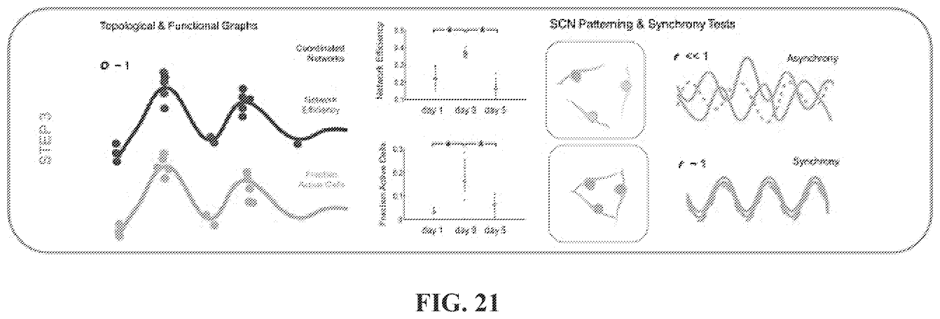

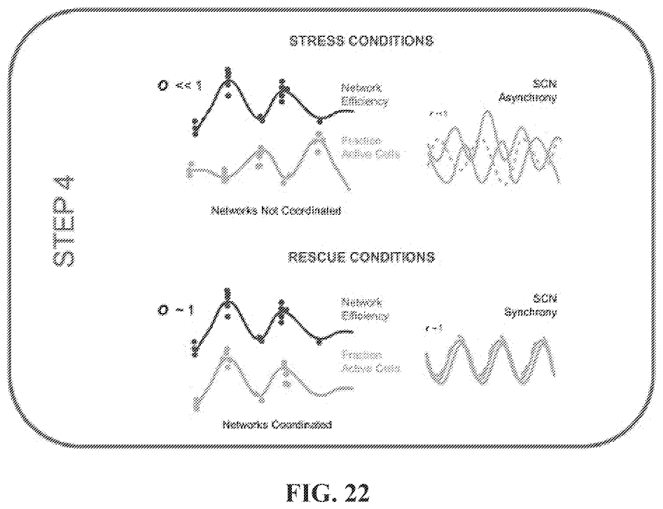

cytoNet is a user-friendly method to analyze spatial and functional cell community structure from microscope images, using the formalism of network science (FIG. 18). cytoNet is available as a web-based interface run on Amazon cloud. Users can choose to analyze image files from their desktops or online servers. Coupled with its ease-of-use, cytoNet's versatility makes it accessible to researchers across domains. The network was originally designed as a modeling approach to study populations of developing neurons (1) and characterize how vascular cells respond to neurotrophic factors (119, 120). The inventors have extended the approach to case studies in a number of other biological systems.

An example representing one embodiment of the methods described herein is described below.
Step 1: Living Neural Networks Assay is developed to image 3D development of human neural stem cells into networks. (A) Day 7 iPSC NCRM-5 neural network. (B) Detection of dendrite length and branching during neurodifferentiation. (C) Graph creation across biological scales and imaging modalities: cell morphology mapped to graph properties (fixed cells); soma (nodes) and dendrites (edges) tracked live.
Step 2: Graphical Analysis of the Functional and Spatial development of the Neural Networks Physiologically active iPSC model. TuJ1, neurons. Fluo-4, Ca2+ signaling dye. DAPI, nuclei. VGAT, inhibitory synapses. NCRM-5 shown. 20×. Schematic of the structure-functional graphs.
Step 3: Functional Checks on Graphical Development Coordination tests (O parameter) between spatial and functional changes in the brain networks Circadian synchrony tests (r parameter)
Step 4: Stress and Rescue Checks on Graphical Development Coordination tests between spatial and functional changes in the brain networks Circadian synchrony tests.

### II. DEEP LEARNING, NEURAL NETWORK(S), AND RELATED PROCESSES

As used herein, “deep learning” refers to the application of learning tasks of artificial neural networks (ANNs) that contain more than one hidden layer. Deep learning is part of a broader family of machine learning methods based on learning data representations, as opposed to task specific algorithms. One key aspect of deep learning is its ability to learn multiple levels of representation of high-dimensional data through its many layers of neurons. Furthermore, unlike traditional machine learning methods, those feature layers are not pre-designed based on domain-specific knowledge and hence they have more flexibility to discover complex structures of the data.

Embodiments of the present disclosure is predicated on a neural network. Accordingly, a neural network and the issues of existing neural network circuits that provide the neural network are described. A neural network is a simulation model of a neural circuit network of living bodies. The neural network performs information processing by using a neuron that simulates a neuronal cell, which is a functional unit of a neural circuit network, as a functional unit and disposing a plurality of neurons in a network form. Examples of the neuron network include a layered neural network having neurons connected in layers and a mutually connected neural network (a Hopfield network) having neurons mutually connected with one another.

A neural network has two primary functions. One is a “processing” function to obtain an output from inputs, and the other is a “learning function” to set a relationship between an input and an output of a whole neural network to a desired relationship.

Processing Function. The operation performed in information processing is described below with reference to a layered neural network. The layered neural network includes the following three layers: an input layer, an intermediate layer, and an output layer. Each of the layers includes at least one neuron. Each of the neurons in the input layer is connected to each of the neurons in the intermediate layer. Similarly, each of the neurons in the intermediate layer is connected to each of the neurons in the output layer. An input signal is input to the input layer and is propagated to the intermediate layer and to the output layer. Thereafter, the input signal is output from the output layer. In the neuron, a predetermined arithmetic operation is performed on an input value, and the output value is propagated to a neuron in the next layer. Accordingly, the output value output from the output layer serves as a final output of the neural network. The above-described series of processes represent the information processing performed by the neural network. If the number of neurons included in the intermediate layer is sufficiently increased, any input and output can be provided. While the layered neural network can include three layers, a plurality of the intermediate layers may be employed.

A neuron that serves as a constituent unit of the neural network is described next. The neuron includes synapse portions and a neuron portion. Note that the number of synapse portions is equal to the number of the neurons connected to the previous stage, that is, the number of input signals. The synapse portion assigns a weight to each of a plurality of input signals input from the outside. The synapse portion assigns a weight to an input signal input from the outside. Each of weights (w1, w2) is called “connection weight”. The neuron portion sums the input signals each weighted by the synapse portion, performs a nonlinear arithmetic operation on the sum, and outputs the result of the operation. Let xi (1, 2, . . . , n) be the input signals from the outside. Then, n is the same as the number of input signals. Each of the synapse portions multiplies the input signal by the corresponding one of the connection weight wi (1, 2, . . . , n), and the neuron portion calculates a sum Vn of the products, as follows:

$${V}_{n} = \sum {w}_{i} {x}_{i} ; \qquad ( 1 )$$

where Σ denotes the summation sign with respect to i.

In addition, the neuron portion performs a nonlinear arithmetic operation f on the obtained sum Vₙ and defines the result as an output value y. Accordingly, the output y of the neuron is expressed as follows:

$$y = f ( {V}_{n} ) . \qquad ( 2 )$$

Note that a monotonically increasing function with saturation is used as the nonlinear arithmetic function f. For example, a step function (a staircase function) or a sigmoid function is used as the nonlinear arithmetic function f.

Since a plurality of the neuron portions can simultaneously perform an arithmetic operation in a neural network circuit, the neural network circuit has a parallel processing property. That is, unlike the sequential information processing performed by an existing computer, the neural network circuit can parallel information processing.

Learning Function. One of the important characteristics of the neural network is that the neural network has a “learning function” in addition to a “processing function” for obtaining an output from an input, as described above. As used herein, the term “learning” refers to setting the relationship between an input and an output of the whole neural network circuit to a desired relationship by updating the connection weight of each of the above-described synapse portions.

The “learning” is primarily categorized into “unsupervised learning” and “supervised learning”. In the unsupervised learning, by inputting input signals to a neural network, a correlative relationship among the input signals input to the neural network is learned by the network. In contrast, in the supervised learning, input signals and a desired output signal corresponding to the input signals are given to a neural network. The desired output signal is referred to as a “teaching signal”. Thereafter, learning is conducted so that an output signal obtained when the input signals are given to the neural network is the same as the teaching signal.

Examples of neural networks include Fully Connected Neural Networks (FCNNs), Recurrent Neural Networks (RNNs), Convolutional Neural Networks (CNNs), Long Short-Term Memory (LSTM) networks, autoencoders, deep belief networks, and generative adversarial networks.

In the layered neural network a learning method called error back-propagation learning is widely employed. The error back-propagation learning is conducted as follows: (1) Samples for learning conducted by a neural network (input signals and a teaching signal) are provided to the neural network. (2) An actual output of the network resulted from the input signals is compared with the teaching signal, and an error between the output of the network and the teaching signal is calculated. (3) The connection weight of each of synapses is adjusted so that the error becomes small. (4) The connection weights are adjusted in order of proximity of the synapse to the output layer (in a direction toward a synapse in the input layer). (5) The above-described steps 1 to 4 are repeated for all the samples. (6) The above-described steps 1 to 5 are repeated for all the samples until the error reaches a predetermined value.

As the name of the algorithm “error back-propagation learning” implies, the error propagates from the neuron in the input layer to the neuron in the output layer during the error back-propagation learning.

Deep learning technology is the technology of performing at least one of learning, determining, and processing on information using an artificial neural network algorithm. An artificial neural network may have a structure of connecting layers to each other and transferring data between the layers. Such a deep learning technology can learn a massive amount of information through an artificial neural network using a Graphic Processing Unit (GPU) optimized for a parallel operation. An example of a deep learning accelerator is one or more relatively specialized hardware elements operating in conjunction with one or more software elements to train a neural network and/or perform inference with a neural network relatively more efficiently than using relatively less specialized hardware elements. Some implementations of the relatively specialized hardware elements include one or more hardware logic circuitry elements such as transistors, resistors, inductors, capacitors, wire interconnects, combinatorial logic (e.g., NAND, NOR) gates, latches, register files, memory arrays, tags for memory arrays, content-addressable memories, flash, ROM, DRAM, SRAM, Serializer/Deserializer (SerDes), I/O drivers, and the like, such as implemented via custom logic, synthesized logic, ASICs, and/or FPGAs. Some of the relatively less specialized hardware elements include conventional CPUs and conventional GPUs.

### III. NEURAL CELLS AND CO-CULTURES

The methods and cells described below illustrate examples of human neural culture or co-culture system. For the generation of various cell types from various types of stem cells (SCs) (e.g., pluripotent stem cells (PS), induced pluripotent stem cells (iPS)), step-wise differentiation protocols can be used based on growth factor timing and growth matrix or surface.

There are various types of stem cells, including pluripotent stem cells, multipotent stem cells, and progenitor stem cells (also called unipotent stem cells). SB-1 cells, SB-2 cells, blastomere-like stem cells (BLSCs), and very small embryonic-like stem cells (VSELs) are pluripotent stem cells. Mesenchymal stem cells (MSCs), multipotent adult progenitor cells (MAPCs), bone marrow derived multipotent stem cells (BMSCs), and multipotent adult stem cells (MASCs) are multipotent stem cells. Neural stem cells, retina stem cells, olfactory bulbs stem cells, epidermal stem cells, muscle stem cells, intestine stem cells, pancreatic stem cells, heart stem cells, liver stem cells, kidney stem cells, endothelial stem cells, adipocyte or adipose-derived stem cells, marrow-isolated adult multilineage inducible (MIAMI) cells, pre-mesenchymal stem cells (pre-MSCs), mesenchymal progenitor cells, hematopoietic progenitor cells (HPCs), multipotent progenitor cells (MPPs), lineage-restricted progenitor cells (LRPs), common myeloid progenitor cells (CMPs), and common lymphocyte progenitor cells (CLPs) are progenitor stem cells.

Glial cells. For the generation of astroglial and oligodendroglial cells from human pluripotent stem cells (hPSCs) a step-wise differentiation protocol through a transient neuroepithelial cell stage is applied. For the differentiation of microglial cells from human pluripotent stem cells a step-wise differentiation protocol through a transient endothelial cell stage is applied. Once differentiated, the astroglial, oligodendroglial, and microglial cells may be combined with other neural cells in a culture system.

Stem cells are differentiated by culture in neural media in the presence of an induction media for a period of time sufficient. The induction media comprises one or more growth factors or agents needed for a desired cell type. As an example, for derivation from human pluripotent stem cells, hPSC colonies are detached as clumps and cultured in bFGF-free human embryonic stem cell medium (hES medium, DMEM/F12 (containing L-Glutamine and Sodium bicarbonate)+20% KSR+Glutamax [2 mM]+NEAA [100 μM]+2-mercaptoethanol [100 μM]+sodium pyruvate) in the presence of an effective dose of a ROCK inhibitor and effective doses of SMAD signaling inhibitors to generate embryoid bodies. These embryoid bodies are then seeded in neural medium on PO/laminin-coated plates to form neuroepithelial cells. Neuroepithelial cells are then detached and cultured in neural medium to form neurospheres. The neurospheres are resuspended in medium with an effective dose of EGF and bFGF to generate astroglial committed spheres which can be resuspended as single cells in neural medium with serum or BMP2/4 and an effective dose of CTNF.

Excitatory neurons. For the generation of excitatory neurons cells from human pluripotent stem cells (hPSCs) a direct differentiation protocol through exogenous expression of neurogenic transcription factors may be used. The hPSC are cultured in the presence of medium and an effective dose of a ROCK inhibitor, and induced to express an effective dose of Ngn2 or NeuroD1, e.g. by lentiviral infection. The cells are cultured, e.g. in neuronal medium, in the presence of an effective dose of a ROCK inhibitor until neuronal differentiation initiates to generate committed immature induced neuronal cells, which can be replated in medium for the neural co-cultures.

Inhibitory neurons. For the generation of excitatory neurons cells from human pluripotent stem cells (hPSCs) a direct differentiation protocol through exogenous expression of neurogenic transcription factors may be used. The hPSC are cultured in the presence of medium and an effective dose of a ROCK inhibitor, and induced to express an effective dose of Ascl1, Dlx2, and Myt1L, e.g. by lentiviral infection. The cells are cultured, e.g. in N3 medium, in the presence of an effective dose of a ROCK inhibitor until neuronal differentiation initiates to generate committed immature induced neuronal cells, which can be replated in medium for the neural co-cultures.

Neural Co-cultures. One or more of the neuronal subtypes described above can be provided in a co-culture. Cells can be plated to achieve the desired combination. The ratio of excitatory/inhibitory neurons may be around about 90:10; 80:20; 70:30; 60:40; 50:50, 40:60; 30:70; 20:80; 10:90; etc. The percentage of excitatory neurons in the combined excitatory/inhibitory neurons is about 10%, 20%, 30%, 40%, 50%, 60%, 70%, 80%, or 90%. The percentage of excitatory neurons in the combined excitatory/inhibitory neurons is from about 10% to about 20%, 30%, 40%, 50%, 60%, 70%, 80% or 90%, from about 20% to about 30%, 40%, 50%, 60%, 70%, 80% or 90%, from about 30% to about 40%, 50%, 60%, 70%, 80% or 90%, from about 40% to about 50%, 60%, 70%, 80% or 90%, from about 50% to about 60%, 70%, 80% or 90%, from about 60% to about 70%, 80% or 90%, or from about 70% to about 80% or 90%, all inclusive. The number of neurons plated may be from about 10⁴, 10⁵, 10⁶ per well or more.

Different cell types of the systems are combined according to the desired phenotypic readout of the application, e.g. modulating effects of compounds on inhibitory neurons in a neuronal network. The neural co-culture system may be of a size appropriate for the assay, typically comprising up to about 5×10⁴, up to about 10⁵, up to about 5×10⁵, about 10⁶, up to about 5×10⁶ neurons, up to about 10⁷ neurons. The neural co-culture may comprise up to about 5×10⁴, up to about 10⁵, up to about 2.5×10⁵, about 5×10⁵ glial cells. The neural co-culture system is grown on a suitable adhesive substrate depending on the detection method used for measuring neuronal activity. Media composition for neural co-culture system may vary in ion content, nutrient, and growth/specification factor supplementation according to applied detection method.

The neural cells can be seeded and maintained on MEA plates, which are specialized tissue culture plates comprising microelectrodes integrated into the well bottom for detection of extracellular currents and local field potentials (see, for example, the Maestro Platform from Axion BioSystems). The culture dishes or MEA plates may be precoated with a suitable substrate, including without limitation laminin, PEI, matrigel, etc.

The neural cells can be seeded and maintained on plates with clear well bottoms, which can be used for image-based analyses. The clear-bottom plates may be precoated with a suitable substrate, including without limitation laminin, PEI, PO, PDL, matrigel, etc.

In one aspect, the in vitro model provides functional and mature human neuronal and/or glial cell cultures or co-cultures capable of forming synapses, neuronal circuits, and neuronal network, the co-culture comprising: in vitro differentiated functional human neuronal cells; and glial cells, such as mouse, rat, or human glia cells.

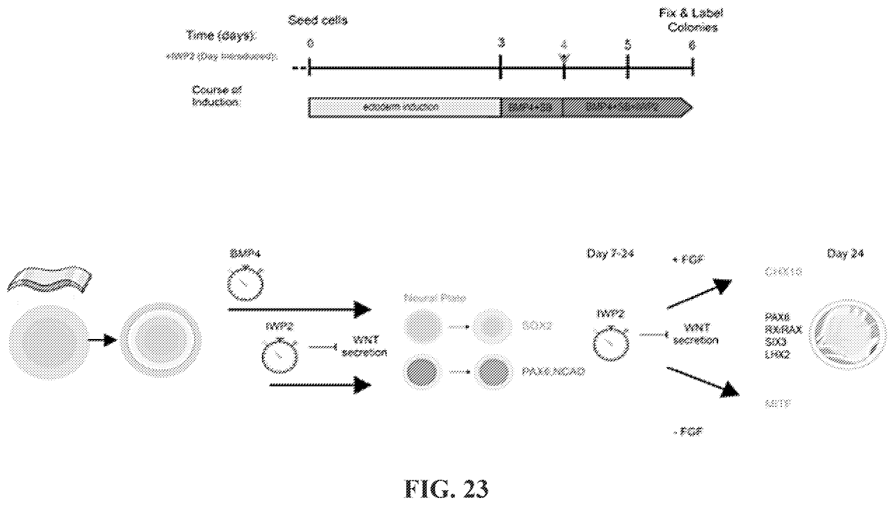

Certain embodiments include, for example, isolating induced pluripotent cells from blood or other tissues or fluids (e.g., isolating the peripheral mononucleated cells) of volunteers. Exposing these or other stem cells to neurodifferentiation protocols, e.g., overall the timing and combination of factors are designed to activate specific transcription factors representative of the SCN (Six3, Six6, Lhx2, Lhx1, VIP, AVP) or eye (BRN3a, KRT12) by inhibiting or stimulating neurodevelopmental pathways (via WNT, BMP)(see FIG. 23).

### IV. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Cell Plasticity as a Function of Daily Activities

The studies described in the following example test whether biomarkers of DNA repair, neuro-secreted extracellular vesicles and/or proteins differentially change in the blood as a function of sleep quality in humans; develops biomimetic models of the eye and suprachiasmatic nucleus specific for individuals with extremes of blood-borne DNA repair and sleep quality; and develops a working, testable model and computational simulations of how brain cells change as a function of rest.

A. Evaluation of Biomarkers of DNA Repair, Neuro-Secreted Extracellular Vesicles and/or Proteins Differentially Change in the Blood as a Function of Sleep Quality

Unique experimental testbed. Daily activity and brain changes are recorded for volunteers whose living “brain models” are being studied in the lab. In addition to elucidating the structure/function of the human system, these models can lay the groundwork for regenerative medicine applications that incorporate living tissue constructs.

AI-graph hybrid models to predict structure from activity. To link the structure/function relationships observed in brain cells, a graph-based framework (1,4) is developed. Here, the graph paradigm is integrated with a convolution neural network to enable improvement of the accuracy of both approaches. The predictive models provide a platform to test numerous conditions that may change the ability of the brain to repair.

Predictions of brain changes across scales (behavior to cells). The methods to predict cell changes from non-invasive recordings enable a way to ‘see’ into the living human brain with minimal risks. Additionally, the models link systems-to-cell level data.

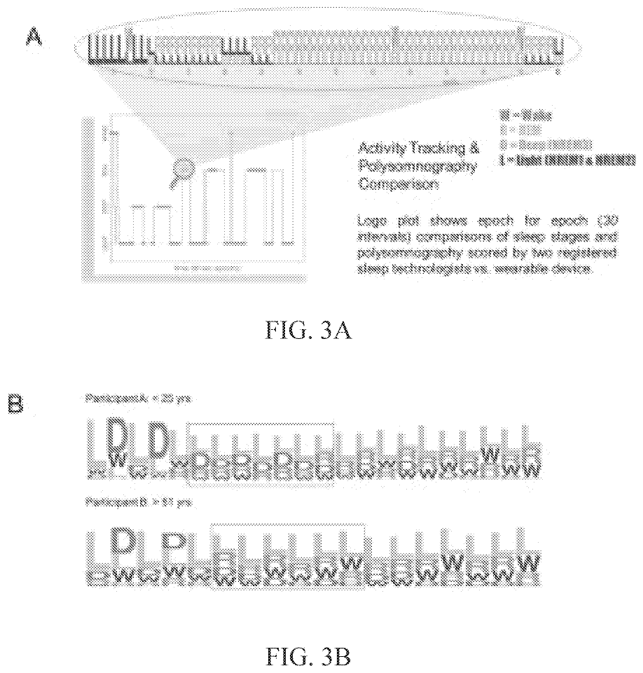

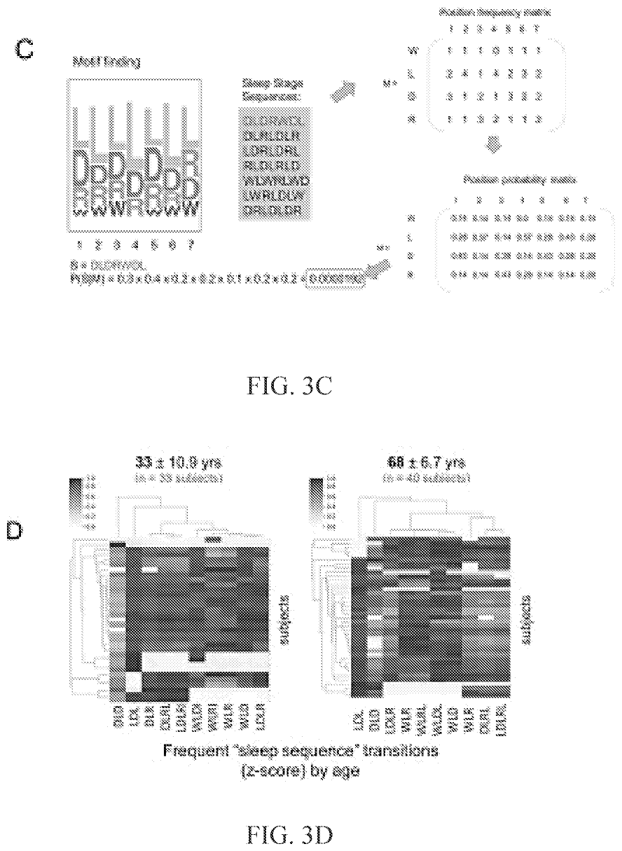

Developing a pipeline to identify quantitative sleep signatures. Sleep signatures can be identified from 200 volunteers three ways: (1) by principle spectral component (PSC) analysis of Dreem headband EEGs (53), (2) cosinor analysis of heartrate data to characterize deviations in circadian rhythm (54-56); and (3) by application of motif finding applied to the recorded sleep data. Motif-finding is common to methods that mine patterns in DNA sequences. The inventors capitalize on these algorithms by substituting nucleic acids (A,T,C,G) with the stages of sleep recorded by the Fitbit™ device (deep, REM, light, wake) to identify frequent sequences (e.g., deep-light-REM). In a first analysis of sensor-based tracking, the sleep motifs were ranked most representative of specific demographics, including age (25) (FIG. 3). Macro sleep properties like REM duration also show age dependency in the subjects, confirming prior studies (53). Sleep stages have been associated with distinct functions like memory consolidation, and transitions in sleep stages manifest in glia and neuronal changes in animal models (57). Disrupted sleep is also characterized by high variability in sleep staging. Shrinkage clustering (39) is applied to the combination of macro features, features identified by PSC and cosinor analysis, and motifs to identify sleep signatures, and group volunteers with similar consistently disrupted or high-quality sleep.

Validation: the inventors calibrated the wearable device data to polysomnogram exams (PSG) recorded in a sleep clinic for 12 individuals with varying degrees of sleep dysfunction (healthy to severe sleep apnea)(58). A recurrent neural network is trained on the paired Fitbit™ and PSG datasets to transform the lower-quality, continuous data to PSG-scored hypnograms from EEGs. This unique approach will allow us to compare findings using wearable devices to analyses performed on public sleep EEG datasets (53).

Screen for biomarkers of DNA repair and neuronal cell changes. Blood samples are obtained for 30 volunteers representative of the sleep signature groups identified above. ELISA screens are run for neural damage (Aβ1-42, P-T181-tau) and repair (BDNF, gpld1) markers in plasma. Volunteers' peripheral blood mononucleated cells (PBMCs) will be isolated and immunostained for the nuclear DNA repair marker 53BP1 (59,60) and isolate extracellular vesicles (61-65) from plasma. Epigenetic sequencing (ATACseq of PBMCs and exosomes) and mass spectroscopy (66,67) is performed.

Test for associations between volunteers' sleep signatures and bloodborne markers of DNA repair and neuronal cell changes. Applying MetaGalaxy pipeline including multivariate analysis, signaling-network decisions trees, clustering, and cluster optimization to the molecular data (39,40,68), is used to test whether blood-borne markers correlate to sleep signatures for the 30 subjects. Temporal changes in blood-borne markers for each volunteer is assessed from blood draws every 6 months, and before and after sleep for 3 days for up to four volunteers representing extremes of sleep quality and DNA repair.

Sleep signatures relate to blood-borne markers of repair or neurogenesis. Blood-borne markers of fatigue, such as creatine kinase, Aβ, cortisol and a variety of other inflammatory markers change with sleep (13,69-71). Qualitatively, disrupted sleep has also been associated with DNA damage (72). At the same time, pioneering work has shown that blood-borne neurogenesis factors change with exercise in humans (73,74). The method to transform wearable device to PSG data and use of Dreem headbands also mitigate data quality risks and allow us to compare our findings to public EEG datasets. These studies define what quality sleep means at a molecular level and lend evidence to the hypothesis that the human brain rejuvenates at the cellular level as function of rest.

B. Develop Biomimetic Models of the Eye and Suprachiasmatic Nucleus Specific for Individuals with Extremes of Blood-Borne DNA Repair and Sleep Quality.

Establish iPSC models of brain regions regulating sleep-wake cycles. Light entrainment of the eye and circadian rhythm control by the retina-innervated SCN govern daily sleep (75). Step 1: Adapting established protocols, an optic cup model is designed to recapitulate the retina and cornea from control NCRM-5 iPSCs (76-79). A fibrin hydrogel is substituted as the matrix within microwells (6,7). The retina is vascularized shortly before birth, and in the accelerated in vitro timeline, the addition of endothelial progenitors is tested at differentiation days 30, 90 and 120. To produce the SCN model, iPSCs are differentiated on micropatterned wells into hypothalamic cells (80,81) and further reprogrammed by day 45 through activation of transcription factors (Six3, Six6, Lhx2, Lhx1)(82-85). In addition to protein markers indicative of eye (e.g., BRN3a, KRT12) and SCN (VIP, AVP) tissue, two key functional readouts are used to validate the brain models: coordination of structural-functional network development and circadian synchrony of the SCN cells, as described below.

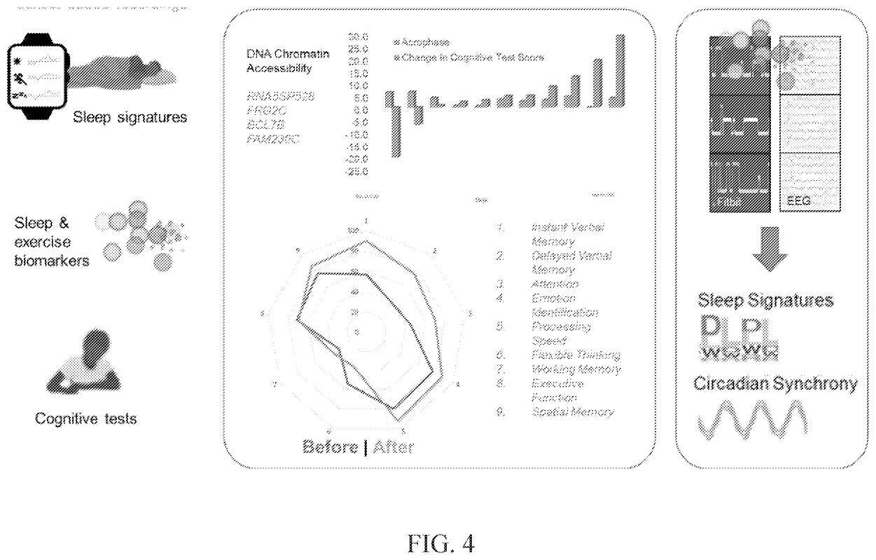

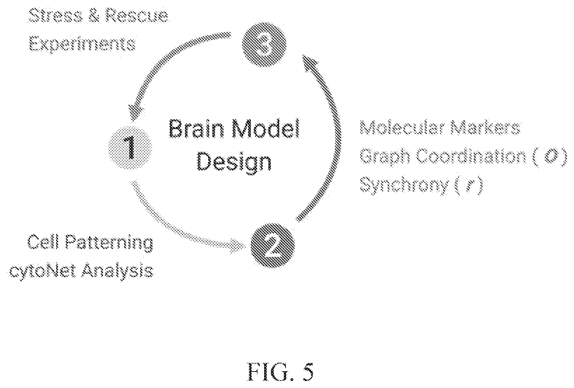

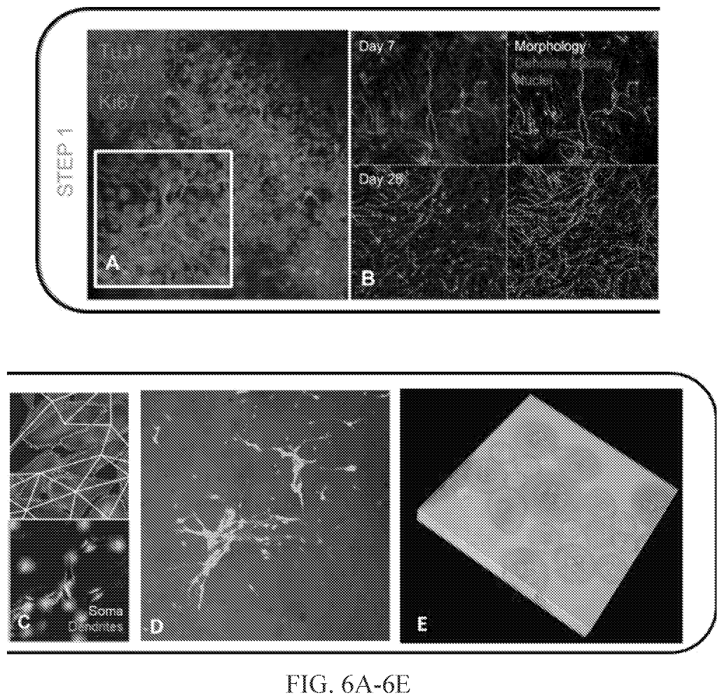

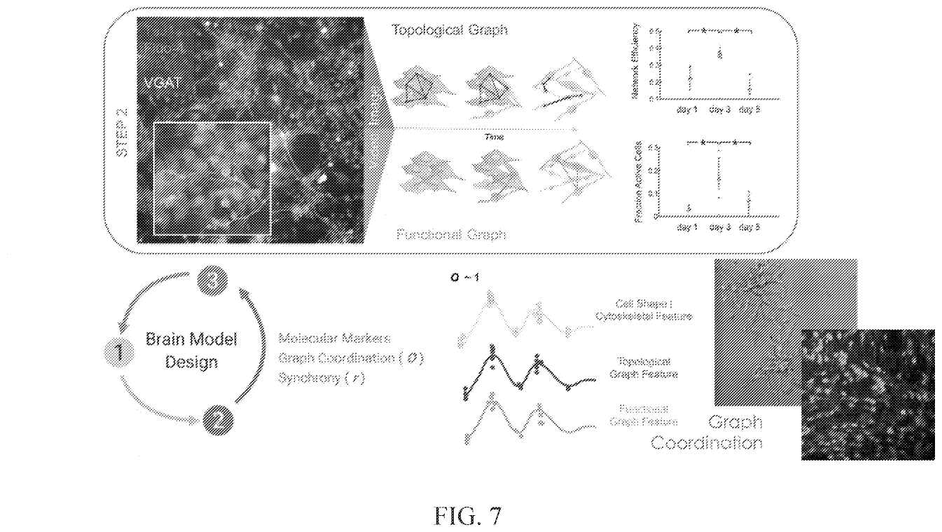

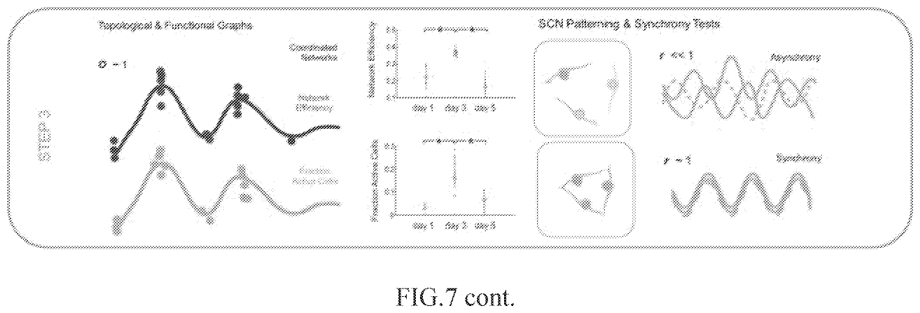

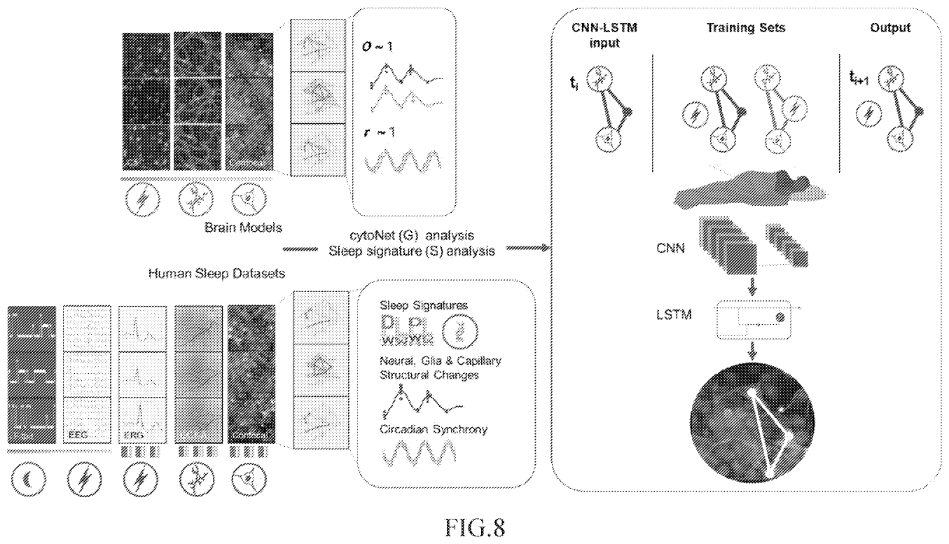

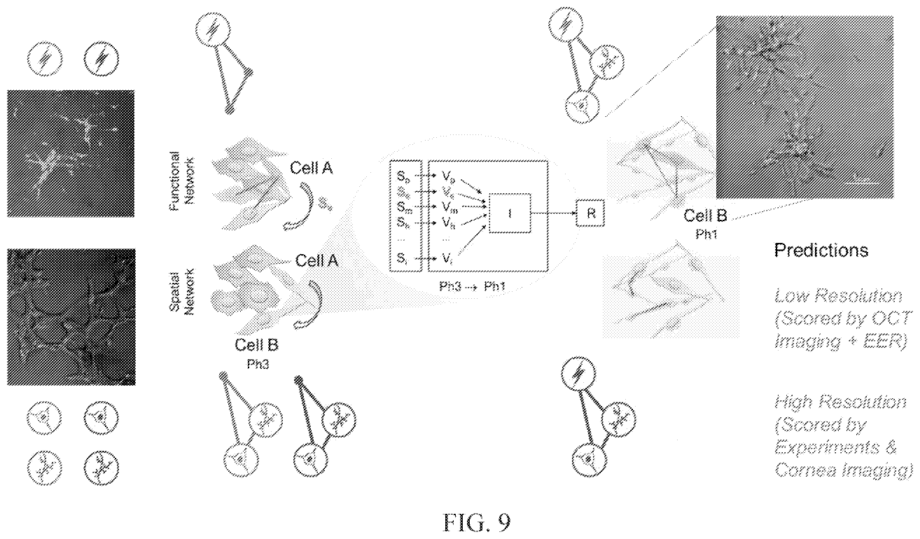

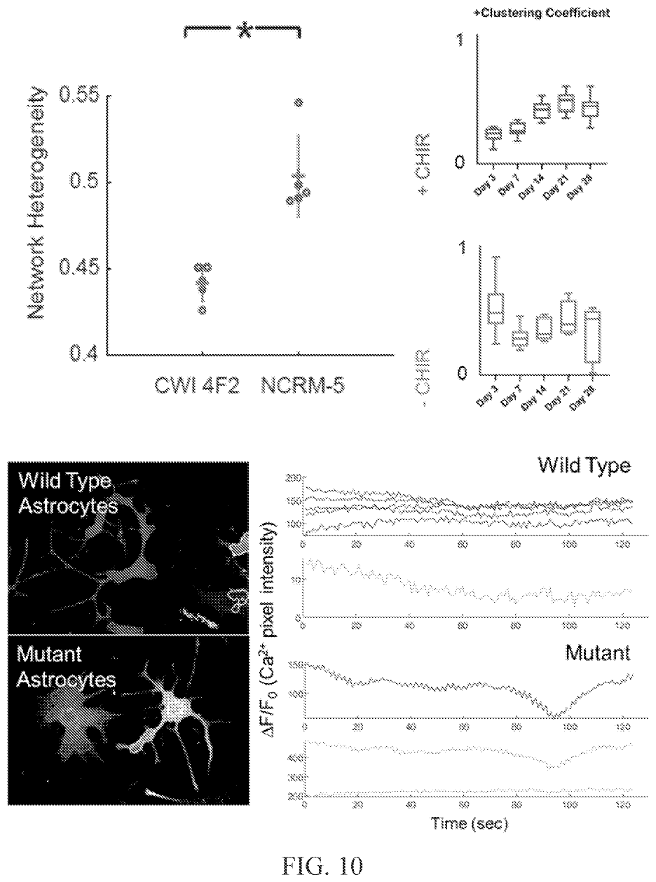

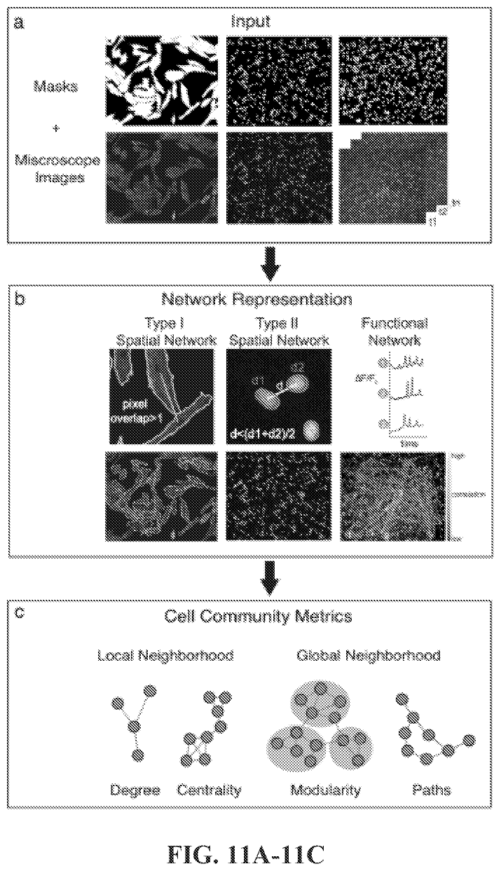

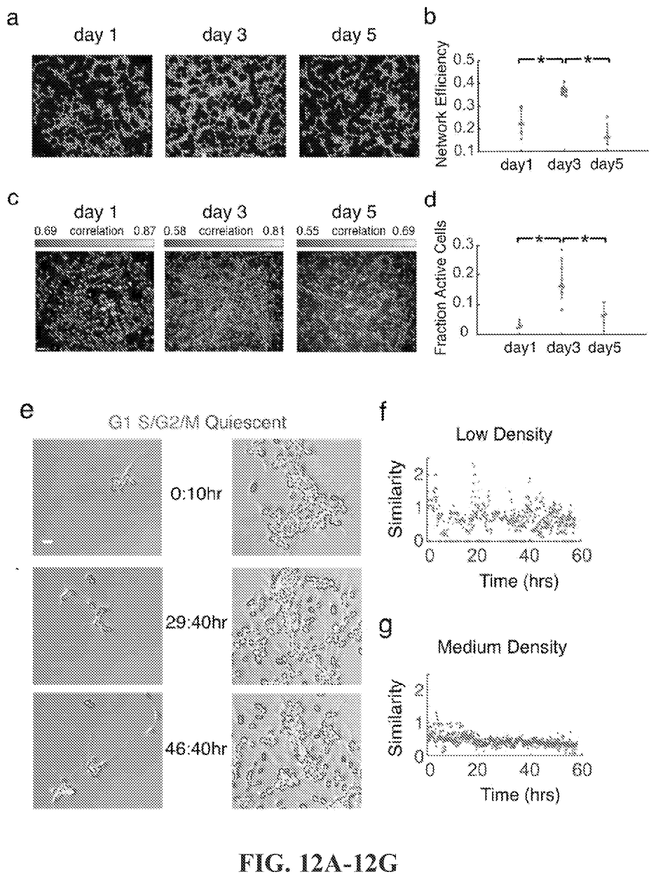

Develop a pipeline to characterize the brain models (FIGS. 4 and 5) Live imaging is performed during and after iPSC differentiation on a dedicated incubated chamber on a confocal microscope. At terminal timepoints of interest, imaging will also be performed in the present of Ca2+ dyes ( 1/40th to 1 sec framerate, 10 min). Samples are fixed and stained for cell type, neurogenesis, gliogenesis, and synaptic proteins. Co-registered images and Ca2+ videos are analyzed by cytoNet. Steps in the cytoNet processing pipeline are: (1) segment cells and characterize cell morphology; (2) create a topological and functional Ca2+ graph, and (3) calculate cell biochemical phenotypes (4, 38, 41). Graphs relate structural, biochemical, and electrical features of brain cells over time (4). Nodes are cells or subparts of cells (FIG. 6C). Edges represent physical or functional connectivity (FIGS. 6F and 6G). Functional connectivity is determined by cross-correlation of the Ca2+ signal between cells (1,86). Step 2: Graph coordination and SCN synchrony is tested in the brain models using cytoNet's output: (1) Graph coordination: the degree to which topological graph features (e.g., network efficiency, 4-star motifs) are coordinated with functional network features is determined. The coordination metric Oi € [0,1] is defined by (cross-correlation+normalized mutual information)/2 between the two time-series. High coordination O˜1 between network efficiency and the fraction of Ca2+active cells was observed in three control lines (ReNCell, hNP, NCRM-5). (2) Circadian synchrony: As single cells, SCN cells have autonomous cyclic expression of Ca2+ activity and circadian clock genes. In tissue and closely packed in culture, SCN cells synchronize activity and clock gene oscillations on a 24-hour cycle. Cosinor analysis is applied to the Ca2+ activity for each SCN cell. Synchrony of the fitted sine waves will be measured by the Kuramoto order parameter r, which equals 1 when phases are identical (min. 0)(87). Volunteers' iPSC models will be designed with the differentiation conditions (e.g., seeding density) that yield the highest O and r for the NCRM-5 iPSCs.

Develop eye and SON models for volunteers with extremes of DNA repair and sleep quality. Volunteers are stratified by Shrinkage Clustering (39) across biometrics from 6 months of data to identify ‘high quality’ and ‘low quality’ sleep groups (25). Extreme is defined by at least two standard deviations (+) from the average frequency of sleep stage motifs, macro sleep properties and/or molecular expression of DNA repair. Volunteers who qualify as extreme are chosen from within a poor sleep quality group. The volunteer with the healthiest sleep profile from five existing iPSC lines is chosen for comparison. Biomimetic assays and non-invasive imaging will be performed for selected volunteers.

Characterize brain model responses to stress and sleep regulators. Step 3: Following differentiation, live imaging is performed continuously for 3 days to study responses of the cornea, retina and SCN tissues to stress. Intermittent hypoxia at 2% O₂, hypo- and hyperglycemia, zolpidem, and transient silencing of PER2 (88,89) to mimic circadian clock disruption is applied, followed by rescue conditions (normoxia, normoglycemia, wash-out of biochemical regulators) at intervals of 4, 8, 12 and 24 hrs (FIG. 6H). Ca2+ imaging; immunostaining for neurogenesis, gliogenesis, and synaptic proteins; and cytoNet analysis are performed.

The inventors established neurogenesis assays and the methods to capture electrical-structural properties of neurons and glia (1). iPSC lines have been derived from five volunteers whose daily sleep has been digitally recorded for >12 months. The inventors patterned an ectoderm (8), whose layers are developmental precursors to the cornea, retina and SCN. Preliminary work (7) and a study in cortical organoids (90) demonstrate feasibility of vascularizing the models. Well-established alternatives to the optic cup are avascular cornea (91-94) and retina iPSC models (76,91,95,96). Commercially available SCN 2.2 cells are an alternative SCN synchrony model. Live reporters for clock genes (97) and Ca2+ can also augment the imaging. The inventors expect that dedifferentiation, synaptic rearrangement and/or genesis events as a function of stress will occur less frequently in the iPSC models from volunteers with consistently poor vs. high quality sleep, and reestablishment of O˜1 and r˜1 is less likely (FIG. 6H).

### C. Develop a Working Theory and Computational Simulations of how Brain Cells Change as a Function of Rest.

Develop a pipeline to characterize the human brain measurements. Eye scans (before/after), electroretinogram recordings (before/after), blood draws (before/after), and EEGs (during) are taken for 3 consecutive nights for the 3 volunteers with extremes of sleep quality. The inventors will image the avascular, highly innervated cornea by confocal imaging (Nidek ConfoScan 4), while the retina is imaged by a handheld fundus scanner and optical coherence tomography (OCT). Corneal images are processed by cytoNet analogously to the biomimetic images. The inventors also adapt cytoNet to the fundus and OCT scans. Nodes become branchpoints of capillaries to enable fiducial markers for comparisons across time (4). Electroretinograms (ERGs), EEGs from Dreem headbands and heartrate data are analyzed by principle spectral component. Circadian synchrony (r) is tested from cosinor analysis of heartrate data across the 3 nights. Blood-borne markers are analyzed as described above.

Design and test methods to predict cell changes during sleep. The inventors integrate a graph-theoretical and convolutional neural network long short-term memory network (CNN-LSTM) modeling (98,99) approach to link the bioassays. Optimizing for time series and spatial data, the CNN-LSTM will be trained to map cellular graph features from the cornea brain model to the graphs of the human cornea images. The AI-graph hybrid model also predicts corneal changes from EEG, ERG; it is then be trained to predict cornea and retina changes from FITBIT™ heartrate and sleep staging data. Training datasets are shown (FIG. 8). Test sets include: (1) 30% of the withheld dataset of paired 3-day sleep recordings (EEG, wearables), eye scans (ERG, confocal, fundus) and molecular screening for three subjects; and (2) images and electrical recordings for stress and sleep regulation assays in the brain models for those same subjects. Model optimization is based on Adam-optimized stochastic gradient descent (100), with Euclidean distance as the similarity metric between predicted and actual graph and cell features. Models are iteratively refined until reaching an accuracy of >80%. The inventors contemplate that dedifferentiation, synaptic rearrangement and/or genesis events occur more frequently in the volunteer(s) with higher quality sleep. Volunteers with poor sleep signatures are predicted to have r<<1 across the three nights, O<<1 in the model systems, and lower blood-borne markers of neurogenesis and repair.

Subtle changes in the cornea and fundus scans are used as a function of sleep signatures. Prior studies elucidated that cornea nerves and retina neurovascular change as a function of neurodegenerative disease, and that sleep can predict neurodegeneration. One aim is to link sleep to cellular brain repair. The environment (e.g., light, position) is less controlled in the human measurements. Conversely, the engineered eye and SCN models lack physiologic constraints present in the human brain. The AI-graph hybrid approach helps address these limitations by (1) providing dual-edge graphs that mathematically link structural relationships to electrical function and (2) mapping in vitro to in vivo by graph features to enable a common set of variables across scales.

### Example 2

### Designing Brain Models by cytoNet Pattern Analysis

### A. Results

The cytoNet pipeline enables the investigation of spatial and functional topology of cell communities in a variety of biological systems. Four case studies are described below.

### Case Study 1: Spatial and Functional Dynamics of Neural Progenitor Cells (NPCs) During Neural Differentiation

An in vitro model of neural differentiation was designed to analyze the dynamics of spatial and functional topology during formation of neural circuits from neural progenitor cells (NPCs)(115). NPCs are known to display structured intercellular communication prior to formation of synapses, which plays an important role in controlling self-renewal and differentiation (121-123). By leveraging the cytoNet method, the inventors sought to capture the dynamic structure of NPC communities and the effect of such community structure on the phenotypes of individual cells.

Data obtained using ReNCell VM human neural progenitor cells is described, in which spontaneous differentiation was triggered through withdrawal of growth factors, leading to rapid cell cycle exit and formation of dense neuronal networks in 5 days (1). Spontaneous calcium activity was captured at days 1, 3, and 5 after withdrawal of growth factors. Following calcium imaging, cells were fixed, and nuclei were stained and reimaged. Nuclei images were then manually aligned by fiducial markers with their corresponding calcium images. The paired image sets allowed the creation of both functional and spatial graphs for the same communities of cells.

Spatial type II graphs showed a rise and fall in global network efficiency during neural differentiation (compared to randomized null models in which edges were rewired while preserving degree distribution). The inventors contemplate that these trends, independently confirmed in multiple NPC lines (1), reflect a transition from topologies favoring global to hierarchical information flow. This possibility was explored through calcium imaging. Functional networks constructed from spontaneous calcium activity revealed network-wide signal correlations, with trends in spontaneous network activity mirroring spatial network parameters. These results suggest that spatial topology predicts functional communication patterns in differentiating NPCs, with high spatial network efficiency at intermediate time points facilitating network-wide communication and low spatial network efficiency at early and late time points mirroring more clustered communication.

Next, the inventors studied the role of cell-cell communication on cell cycle regulation of NPCs. Cell cycle regulation in NPCs is of interest as it has implications for the genetic basis of brain size in different species (124) and aberrant regulation can cause diseases like microcephaly (125). Studies in the ventricular zone of the embryonic mouse neocortex have shown that clusters of clonally-related neural progenitor cells go through the cell cycle together (126, 127). However, it is unclear whether this community effect is a ubiquitous feature of neural progenitor cells. To this end, the cytoNet workflow was employed to determine whether cell cycle synchronization is a feature of differentiating NPCs cultured in vitro.

For this part of the investigation, ReNCell VM human neural progenitor cells were stably transfected with the FUCCI cell cycle reporters (128) to generate Geminin-Venus/Cdt1-mCherry/H2B-Cerulean (FUCCI-ReN) cells. Time-lapse movies of FUCCI-ReN cells were captured after withdrawing growth factors to induce differentiation and built network representations from nucleus images. Adjacency was determined by comparing centroid-centroid distance to a threshold (type II graphs).

To evaluate spatiotemporal synchronization in cell cycle, for each individual cell in a frame, the average fraction of neighboring cells in a similar phase of the cell cycle (G1 phase—mCherry+ and S/G2/M phases—Venus+) was evaluated, normalized by total fraction of that cell type in the population. The average value of this fraction across all cells in an image is called the neighborhood similarity score, Ns. Groups of cells in the low-density culture moved through the cell cycle in unison, which was reflected in periodically high values of the neighborhood similarity score. In contrast, the composition of cell clusters in the medium density culture was relatively heterogeneous, resulting in relatively low values of the neighborhood similarity score over time. Neighboring cells in very low-density cultures are likely to be derived from the same clonal lineage, which explains the high level of synchronization in these cultures (126). This example highlights how cytoNet can be used to derive insight into the role of cell-cell interactions on dynamic cell behavior.

### Case Study 2: Dynamics of Coupled Functional & Spatial Analysis In Vivo

In vivo calcium analysis is an avenue for exploring and understanding the role that individual cells of the nervous system play in processing external stimuli including pain. Pain is mainly mediated by a subset of primary sensory neurons known as nociceptors in Dorsal Root Ganglia (DRG) and Trigeminal Ganglia (TG) (129). How DRG neurons function at a population level under physiological and pathological conditions is unknown. Imaging methods developed to record from hundreds to thousands of neurons simultaneously in the brains of live mice are helping elucidate this (130, 131). To investigate population characteristics of pain-sensing neurons, cytoNet is used to evaluate spatial and functional networks from calcium image sequences obtained in a mouse DRG model.

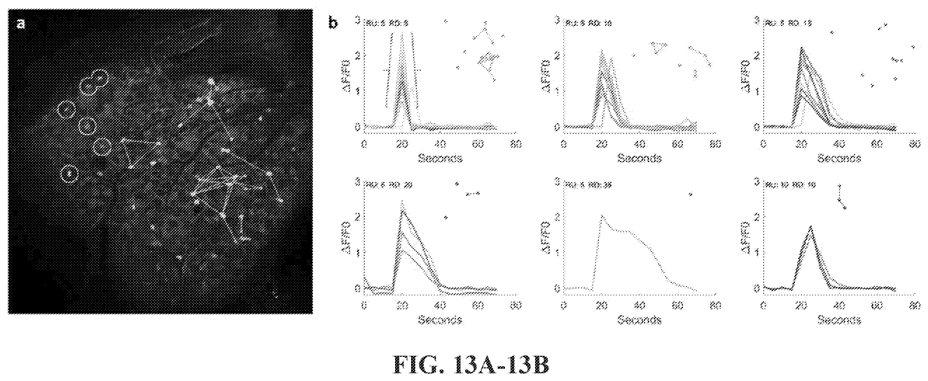

Calcium image sequences, along with single masks identifying individual cells, were inputs to cytoNet (see Methods for details on generation of masks) (FIG. 13). Sensory stimulation experiments produced a single, major signal spike in each segmented cell (130). Measurement of the magnitude (ΔF/F0) of each spike is sensitive to the quality of segmentation; to mitigate this, each cell was characterized not by its spike magnitude, but by the time a cell took to reach its peak value from 20% of that value (ramp-up) and the time needed for the signal to return to 20% (ramp down). Inspection of 44 segmented cells revealed 6 unique combinations of ramp-up times and ramp-down times (FIG. 13b). Ramp-up times were either 5 or 10 seconds while ramp-down times varied between 5 and 35 seconds. This categorization of cells according to functional similarity was combined with the spatial graph of the segmented cells to identify spatial patterns of cells with similar behavior (FIG. 13a). In addition, although the vast majority of segmented cells reached their peak intensity at 20 seconds, a small group of cells along the left side of the tissue peaked at 25 seconds suggesting a right-to-left wave of response (FIG. 13). This case study highlights the utility of cytoNet in analyzing spatial patterns of neural populations with unique functional signatures in an in vivo model.

### Case Study 3: Disentangling the Effect of Cell Community and Growth Factor Stimulation on Endothelial Cell Morphology

In a second application to studying human cells in vitro, cytoNet was used to evaluate the relative influence of local neighborhood density and growth factor perturbations on endothelial cell morphology. From a regenerative medicine perspective, studying the morphological response of endothelial cells to neurotrophic stimuli can help assess the cells' potential angiogenic response following brain injuries that induce the secretion of neurotrophic factors, like ischemic stroke or transient hypoxia (132, 133). Common high-throughput angiogenic assays focus on migration and proliferation as the main cell processes defining angiogenesis, or the growth of new capillaries from existing ones (134). Distinct morphology and cytoskeletal organization of endothelial cells indicate the cell's migratory or proliferative nature, and hence their angiogenic contribution within a sprouting capillary (135). Reproducibly quantifying the morphological response of endothelial cells to neurotrophic factors would enable more targeted approaches to enhancing brain angiogenesis.

An image-based approach to this problem was used, building a library of immunofluorescence images of human umbilical vein endothelial cells (HUVECs) stained for cytoskeletal structural proteins (actin, α-tubulin) and nuclei, in response to various combinations of vascular endothelial growth factor (VEGF) and brain-derived neurotrophic factor (BDNF) treatment. Cell morphology was annotated using 21 metrics described in our previous study (41) (Table 4), which included cell shape metrics like circularity and elongation, and texture metrics for cytoskeletal stains such as actin polarity, smoothness etc. Network representations were designated based on shared cell pixels (type I graphs) and local network properties were described using the metrics in Table 3.

TABLE 3
Local neighborhood metrics calculated at the individual cell level

| Graph Metrics | Symbol | Definition |
|---|---|---|
| Degree | k | Number of neighbors one link away from cell of interest |
| Average Neighbor | kₙ | Average degree of all neighboring cells |
| Degree | | |
| Clustering | C | Number of edges in local neighborhood of a cell, |
| Coefficient | | divided by total possible connections |
| Local Efficiency | Eₗ | Average shortest path length in local neighborhood |
| Node Closeness | cₙ | Sum of reciprocal distances in number of links |
| Centrality | | to all other nodes |
| Node Betweenness | wₙ | Number of shortest paths that pass through a node |
| Centrality | | |
| Shared Cell | S_{b} | Total number of pixels shared with neighbors |
| Border¹ | | |

TABLE 4
Metrics used to define endothelial cell morphology.

| Metrics | Definition | Mathematical Representation |
|---|---|---|
| Cell Size | Cell Spread Area | A_{c} |
| Circulanty | Shape factor | $\frac{4 π {A}_{c}}{{P}_{c}}$ |
| | | Where P_{c} is perimeter of cell |
| Elongation | Shape factor | $\frac{{P}_{c}}{{A}_{c}}$ |
| Polarity* | Distance between center of mass of stain and the centroid of the cell | $\sqrt{{( {χ}_{C , x} - {Ω}_{S , x} )}^{2} + {( {χ}_{C , x} - {Ω}_{S , x} )}^{2}}$ |
| | | Where χ_{c} is centroid of cell and Ωₛ, is center |
| | | of mass of stain |
| Mean* | First moment of grayscale stain intensity distribution | ${\sum }_{i = 0}^{255} \frac{i}{255} \cdot p$ |
| | | Where p is the histogram counts of the image |
| | | fox pixel intensities, with 256 possible bins |
| | | for a grayscale image |
| Standard Deviation* | Second moment of grayscale stain intensity distribution | $\sqrt{{\sum }_{i = 0}^{255} {( \frac{i}{255} )}^{2} \cdot p}$ |
| Third Moment* | Third moment of grayscale stain intensity distribution | $\frac{1}{{255}^{2}} \sqrt{{\sum }_{i = 0}^{255} {( \frac{i}{255} )}^{2} \cdot p}$ |
| Saroothneas* Entropy from Histogram* | Measure of smoothness of stain Measure of randomness of the stain intensity | $1 - \frac{1}{1 + ( \frac{1}{{255}^{2}} \sqrt{{\sum }_{i = 0}^{255} {( \frac{1}{255} )}^{2} \cdot p} )} - \sum p \cdot {log}_{2} ( p )$ |
| Uniformity* | Sum of squared elements in | ΣP² |
| | the histogram counts of the | |
| | image for pixel intensities | |

*Computed for all 3 stain

TABLE 5
Cohen's d effect size for treatment conditions
on morphology metrics shown in FIG. 4

| | | Cohen's d Effect Size | | | |
|---|---|---|---|---|---|
| Morphology | Treatment | No correction | | Correction applied | |
| Metric | Condition | for network metrics | | for network metrics | |
| Cell Size | | 6 hr | 12 hr | 6 hr | 12 hr |
| | | (uncorrected*) | (uncorrected) | (corrected**) | (corrected) |
| | BDNF50 | 0.256 | 0.217 | 0.148 | 0.170 |
| | VEGF50 | 0.151 | 0.023 | 0.093 | 0.068 |
| Mean Actin | | 6 hr | 12 hr | 6 hr | 12 hr |
| | | (uncorrected) | (uncorrected) | (corrected) | (corrected) |
| | BDNE50 | 0.381 | 1.020 | 0.091 | 0.873 |
| | BDNF100 | 0.517 | 2.522 | 0.260 | 1.959 |
| | VEGF50 | 1.121 | 1.018 | 0.348 | 0.740 |
| | VEGF100 | 1.267 | 2.269 | 0.284 | 1.808 |

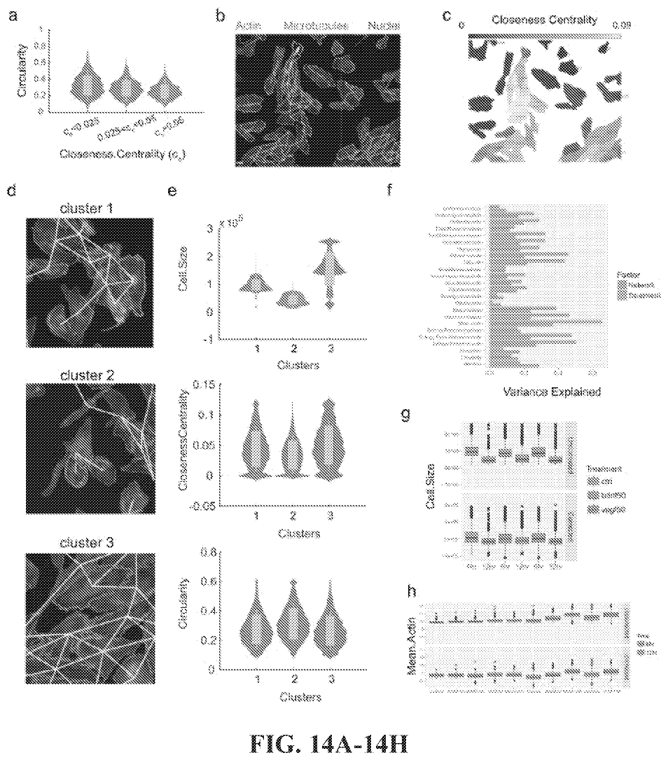

First, density-dependent effects on endothelial cell morphology were quantified in control cultures (without any growth factor perturbation). The analysis showed correlations between cell morphological features and local network properties (FIG. 18). Some of these relationships were expected, for instance the positive correlation between shared cell border and cell size. Other relationships, such as the negative correlation between cell circularity and closeness centrality, capture intuitive notions of the influence of cell packing on morphology (FIG. 14a-c). The closeness centrality of a cell (Table 3) describes its relative position in a colony—cells in the middle of a colony will have higher centrality values than cells at the edge of a colony or isolated cells. The negative relationship between circularity and closeness centrality implies that isolated cells and cells located at the edge of colonies are more likely to have a circular morphology, while cells located at the center of colonies tend to be less circular (FIG. 14a-c). Thus, the analysis revealed that local network properties have a quantifiable effect on cell morphology.

To determine dominant cell phenotypes, cluster analysis was performed on the dataset consisting of 25,068 cells. This analysis revealed 3 major categories of endothelial cells, with unique morphological and network signatures (FIG. 14d-e). Cluster 1 comprised cells with migratory features, including low circularity and intermediate centrality indicative of their position at the edges of colonies. Cluster 2 contained small, circular cells with low centrality indicative of their isolation. Cells in cluster 3 showed proliferative features with large noncircular shapes, and high centrality indicating their positions in the center of colonies. Through this cluster-based phenotyping, the inventors show how cytoNet can be used to infer the local environment and topological arrangement of distinct cell categories within a culture.

Next, a workflow was developed to analyze the effect of growth factor treatments on cell morphology, while correcting for the effect of local network properties. The inventors did this to infer the independent effects of chemical perturbation and local cell crowding on cell morphology. First, a quantile multidimensional binning approach (136, 137) was applied to calculate the variance in morphology metrics that could be individually explained by all local network metrics and growth factor treatments (FIG. 14f). Values were calculated for each morphology metric after correcting for the effect of local network metrics (see Methods). The raw and network-corrected values for two metrics, cell size and mean actin intensity, are shown in FIG. 14g-h. The influence of network properties can be clearly seen on cell size, where at 6 hours, large cell sizes are seen in the uncorrected but not corrected plots (FIG. 14g). The effect of growth factor treatment can be clearly seen in network-corrected mean actin intensity (FIG. 14h), where VEGF and BDNF treatment have dose-dependent effects on mean actin intensity independent of cell crowding effects. Thus, this case study demonstrates the utility of cytoNet in detecting the independent effects of local cell crowding and growth factor perturbations on cell morphology.

### Case Study 4: Spatial Analysis of the Pericapillary Niche in Adipose Tissue

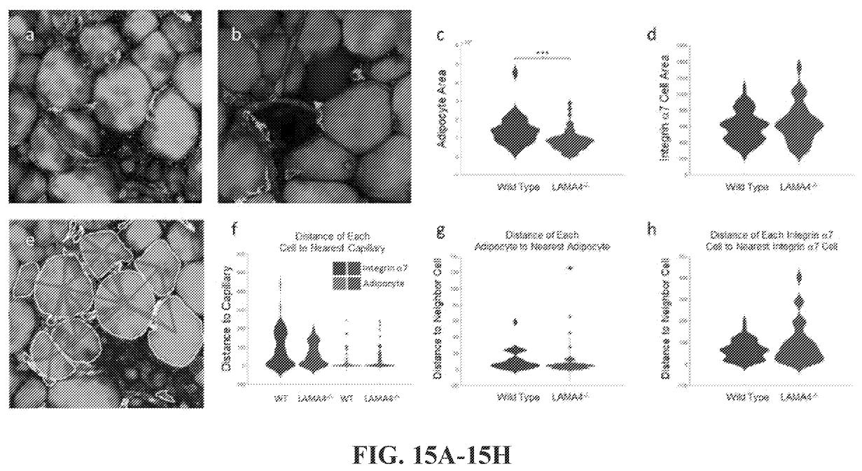

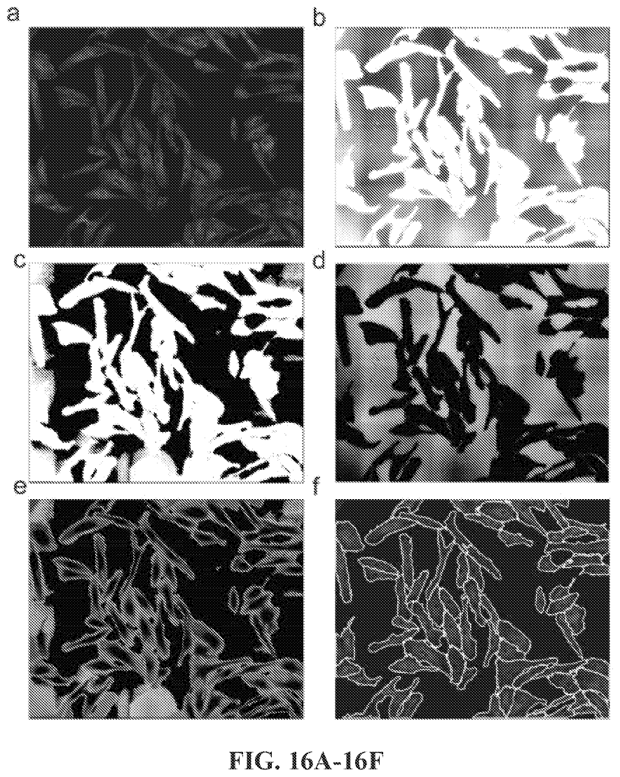

In a second illustration of cytoNet's utility to analyze intact tissue, cytoNet was used to characterize the pericapillary niche within adipose tissue. Specifically, it was sought to understand the role of laminin α4, an extracellular matrix glycoprotein, in adipose tissue. Mice with a null mutation in the laminin α4 gene exhibit resistance to obesity and enhanced insulin sensitivity (138, 139). Understanding how the deletion of laminin α4 affects the spatial distribution of cells present in the adipose tissue can provide insight into the mechanisms underlying the functional change, and guide biomimetic models of the adipose perivascular niche (105, 140, 7). In this Case Study example, the confocal images of adipose tissue and capillaries were segmented by manual tracing on the computer and provided as input to cytoNet. Because blood vessels have noncircular shapes, the distance between the centroids of vessels and other objects may not give a good sense of proximity. As an alternative graph-generation approach, cytoNet can compute the minimum distance between object perimeters to define graph edges. The resulting cell-to-cell perimeter distance table and cell area computations were used to determine differences between wild-type and knockout cells (FIG. 15). The observed adipocytes stained with the BODIPY lipid dye tended to be smaller in knockout tissue compared to wild type (FIG. 15c). This characterization is consistent with the observation that adipose in knockout mice is more similar to beige adipose tissue. In addition, numerical differences were observed in the “distance to capillary” metric for integrin a7 expressing cells between the laminin α4 knockout and wild-type mice models (FIG. 15f), though for the limited sample size they were not statistically significant. Overall, these observations align with findings that the absence of laminin α4 leads to changes in stromal cell structure and distribution in pericapillary niches within adipose tissue (105). The resulting data can be used to guide studies into understanding the mechanisms underlying the effect of laminin α4 on adipose tissue function. Thus, this case study demonstrates the utility of cytoNet in detecting regional variations of cell structure within tissues and in addressing testable spatial hypotheses about tissue function.

Advances in in situ profiling techniques have led to the generation of highly multiplexed imaging datasets describing tissue architecture in great spatial detail (107, 111-115). Spatially detailed imaging datasets have led to a proliferation of computational pipelines designed to test spatially driven biological hypotheses (Table 1). However, many of these analysis pipelines are designed specifically for spatial molecular expression data and are not generalizable to data obtained from other microscopy techniques. Further, due to their reliance on specialized scripts, many pipelines are not readily accessible to biological researchers without programming background.

cytoNet is a user-friendly pipeline for investigation of spatial hypotheses in cell and tissue-based biological experiments. cytoNet is available through an intuitive web interface, eliminating the need to download and install software. Source code is also provided as MATLAB scripts for more advanced users. Pre-segmented masks provided as input to cytoNet are used to build network representations of spatial topography. Accompanying fluorescence or confocal images are used to extract single-cell features and functional relationships. Lastly, network descriptors are combined with single-cell features to explore cell community effects on cell phenotypes.

The utility of cytoNet is demonstrated through four case studies described above. The inventors harness an in vitro model of neuronal network formation from neural progenitor cells (NPCs) to demonstrate a rise and fall in network efficiency during neural differentiation. Accompanying functional network analysis through calcium imaging shows that these trends in community structure likely reflect a transition from global to hierarchical communication during the formation of neural circuits. The inventors further use local neighborhood measures to explore the effect of cell community on cell cycle regulation, showing a density-dependent effect on cell cycle synchronization.

The second case study showed cytoNet's capability for analyzing time-varying functional image sets. In this case, the inventors characterized spatiotemporal calcium signaling recorded from intact brain tissue. Networks can be constructed based on the similarity of temporal behaviors of cells. The combination of the functional networks and spatial networks reveals local groups of cells with similar behaviors and assists in the development and testing of hypotheses of functional subsystems in neuronal tissue.

The inventors also explored the differential effects of cell density and growth factor stimulation on human endothelial cells using cytoNet. By applying unsupervised clustering approaches on a suite of cytoNet-generated metrics describing cell morphology and local neighborhood, the presence of three cell phenotypes were shown. These phenotypes reflect different cytoskeletal states and multicellular interactions indicative of collective behaviors like migration and proliferation. Further, a quantile multidimensional binning approach was leveraged to investigate the differential effects of cell density and growth factor perturbations on cell morphology. This workflow can be used to comprehensively characterize the response of cells to chemical perturbations and aid in drug discovery. Case Study 4 illustrated another translational application of cytoNet: this time to study the effect of an extracellular matrix protein on the phenotype of adipose cells within perivascular niches.

Notably, two of the case studies were applied in vitro to human cells, and two were applied to in vivo image sets. Case Study 1 and 2 capitalized on cytoNet's ability to integrate functional and structural graphs across time in a single mathematical framework. The other two cases illustrated the how cytoNet can be applied to optimize cell phenotyping (Case Study 3 and 4). All of the cases show how cytoNet can help guide hypotheses, inform biomimetic models or tailor therapeutic interventions that reflect a cell's microenvironment.

The network model utilized by cytoNet is a versatile modeling framework that can be used to incorporate many hypotheses on cell-cell interactions and their role in cellular behavior. In future iterations, this framework can be expanded to incorporate non-binary interactions through weighted networks, shift the focus from individual nodes to motifs through simplicial complexes, and include dynamic reconfiguration of networks over time through multilayer networks. Further, once graphs have been defined, graph theory affords a rich array of metrics that can be used to probe network structure, only some of which were studied here. These include a variety of null graph models that can be used to test specific spatial hypotheses.

In summary, the cytoNet method provides a user-friendly spatial analysis software, leveraging network science to model spatial topography and functional relationships in cell communities. This framework can be used to quantify the structure of multi-cellular communities and to investigate the effect of cell-cell interactions on individual cell phenotypes.

### B. Methods

Software. cytoNet is available as a web-based interface at URL www.QutubLab.org/how and associated scripts are available at URL github.com/arunsm/cytoNet-master.git.

cytoNet image analysis pipeline. The cytoNet pipeline begins with masks and accompanying microscope images. The microscope images may be any color or gray-scale based microscopy images (e.g., immunofluorescence, confocal) or a sequence of calcium images (FIG. 18a). The provided mask is used to extract features of cells and to construct spatial and functional graphs (FIG. 18b). Spatial graphs are created by having nodes represent mask objects and edges determined by object distance. Edges can be found by one of two methods for spatial graphs: by evaluating the distance between cell boundaries (type I graphs), or by evaluating the proximity of cells in relation to a threshold distance (type II graphs) (FIG. 18b). The type I graphs are useful when detailed information of cell boundaries and morphology is available, such as in the case of membrane stains or cells stained for certain cytoskeletal proteins. The type II graphs work well with images of cell nuclei, where detection of exact cell boundaries is not possible. In both approaches, cells deemed adjacent to each other are connected through edges, resulting in a network representation. If calcium imaging sequences are provided as input, a functional graph is created based on correlations among calcium time series of different mask objects (FIG. 18b).

Image Segmentation. Image segmentation—the identification of salient foreground objects such as cells—is often the first step in image analysis. The cytoNet pipeline works with pre-segmented masks of images and accompanying microscope images. For users who do not have mask files, cytoNet includes basic image segmentation algorithms including thresholding and watershed operations to generate these masks. The segmentation algorithms included in cytoNet can be parameterized to work well for images with clear delineation of nuclei and cell borders, like the endothelial cell examples provided on the cytoNet website. The cytoNet code also provides frequency detection of cells, where a change in a functional marker (e.g., Ca2+ or FUCCI) delineates cell location. For object detection in most other image sets, the user is referred to programs that focus on cell segmentation (141-143). Multiple research teams have made significant inroads into designing generalizable image segmentation algorithms, among them classic thresholding and watershed approaches (144), pixel based classifiers (141) and more recently deep learning approaches (107, 142, 143). These programs generate masks as output. Users may wish to implement them prior to analyzing community structure through cytoNet. Image segmentation and graph creation are handled separately by cytoNet, enabling flexibility for the user.

Generation of spatial networks. Type I graphs are generated as follows. Mask boundaries are expanded by 2 pixels and overlap of expanded masks is used to assign edges and build an adjacency matrix. Cells touching the image border are included in calculations of local network properties (Table 3) for cells not touching the boundary but are excluded for the construction of the adjacency matrix. Type II graphs are generated as follows: for each pair of objects (nuclei), a threshold distance for proximity is defined as the average of the two object diameters, multiplied by a scaling factor (S). If the Euclidean distance between the object centroids is lower than the threshold distance computed, the pair of objects is connected with an edge. A default scaling factor of S=2 was chosen for the analyses, through visual inspection of cell adjacency.

Generation of functional networks. Functional networks are created using the method described by Smedler et al, (145) where cross covariance between signals is used to assign functional connections between pairs of cells (FIG. 18b). A randomized dataset is generated by shuffling each signal in the original dataset at a random time point. The 99th percentile of cross-covariance values for the randomized dataset is used as a threshold for determining significant correlations.

Network Metric Computation. For both spatial and functional graphs, connectivity is denoted mathematically using an adjacency matrix, A, where Aij=1 if there exists an edge between cells i and j, and 0 otherwise. This concise representation of hypothesized interactions among cells can be used to generate multiple descriptors at a local level for individual nodes and at a global level for the entire graph (FIG. 18c). Extracted metrics are used to visualize and analyze local neighborhood effects on individual cell phenotypes (Table 3), as well as global cell community characteristics (Table 6). Examples of local metrics are number of connections (degree) or notions of centrality, such as ability to act as a bridge between different cell communities (betweenness centrality). Examples of global metrics include measures of modularity such as the number of connected components, and measures of information flow such as path length. All the network metrics described in Table 3 and Table 6 were computed using custom-written code, building upon routines provided in (145).

TABLE 6
Global graph metrics and their normalization to account for network size.

| Graph Metrics | Symbol | Definition |
|---|---|---|
| Node Count | n | Number of nodes |
| Edge Count | m | Number of edges |
| Fraction | A | Fraction of total surface area in |
| Area Cells | | field of view covered by cells |
| Average Degree | avgeK | Average number of connections for a |
| | | node in the network |
| Variance in Degree | varK | Variance of node degree sequence |
| Network | NetworkHeterogeneity | Standard deviation of node degree sequence |
| Heterogeneity | | divided by mean of degree sequence - reflects |
| | | tendency of network to contain hub nodes |
| Average Neighbor | avgeNeighborK | Average degree local neighborhood, |
| Degree | | averaged across all nodes |
| Variance in | varNeighborK | Variance of the average neighbor |
| Neighbor Degree | | degree sequence |
| Network Efficiency | E | The average reciprocal of shortest path |
| | | length across all pairs of nodes, E |
| Average Clustering | C | Fraction of total possible links among |
| Coefficient | | the neighbors of a node that are |
| | | actually across all nodes, C |
| Number of connected | nConnectedComponents | Number of disconnected sub-graphs in main graph |
| components | | |
| Average Size of | avgeComponentSize | Average number of nodes in each connected component |
| Connected Components | | |
| Variance in size of | varComponentSize | Variance in component size sequence |
| connected components | | |
| Network Diameter | networkDiameter | Longest shortest path length of network |
| Isolated Node Count | nIsolatedNodes | Number of nodes with no neighbors |
| Pair Node Count | nPairNodes | Number of independent pair of nodes |
| Triangular loop count | nLoops3 | Number of loops of 3 nodes |
| 4-star motif Count | nStar4 | Number of star mofits with one hub and three spokes |
| 5-star motif count | nStar5 | Number of star mofits with one hub and four spokes |
| 6-star motif count | aStar6 | Number of star mofits with one hub and five spokes |
| Rich-Club | avgeRichClubMetric | Measure of the tendency of nodes with high number |
| Metric Average | | of links to be well connected among each other (71); |
| | | Computed for threshold degrees between 1 and (n-1) |
| Rich-Club | varRiceClubMetric | Variance in rich-club metric for |
| Metric Variance | | thresholds from to (n-1) |
| Assortativity | Assortativity | Pearson correlation coefficient of degrees |
| | | between pairs of linked nodes(72). |

N = number of nodes,
m = number of edges

Cell Culture. Human umbilical vein endothelial cells (HUVEC) were obtained from Lonza and cultured in EBM-2 medium (Lonza) supplemented with penicillin-streptomycin (Fisher Scientific) and EGM-2 SingleQuot bullet kit (Lonza). For imaging experiments, cells were cultured for different periods (6, 12 or 24 hours) in different combinations of vascular endothelial growth factor (VEGF, human recombinant; Millipore) and brain-derived neurotrophic factor (BDNF, human recombinant, Sigma-Aldrich). Concentrations used were in the range 50-100 ng/ml. Controls were the same culture period without growth factor treatments.

Immortalized human neural progenitor cells derived from the ventral midbrain (ReNCell VM) were obtained from Millipore. Cells were expanded on laminin-coated tissue culture flasks, in media containing DMEM/F12 supplemented with B27 (both Life Technologies), 2 μg/ml Heparin (STEMCELL Technologies), 20 ng/ml bFGF (Millipore), 20 ng/ml EGF (Sigma) and penicillin/streptomycin. For differentiation experiments, cells were cultured in medium lacking bFGF and EGF.

Dorsal Root Ganglion Mouse Model. Dorsal laminectomies were performed on anesthetized mice exposing the dorsal root ganglia in the spinal L5 region. The spinal columns were stabilized under a laser-scanning confocal microscope. Stimuli were applied to the hind paw in one of four ways: (1) pressure (rodent pincher analgesia meter), (2) gentle mechanical stroke (brush or von Frey filament), (3) thermal stimuli (immersion in hot or cold water), (4) chemical stimuli (KCl, capsaicin, or TRPV1 agonist applied subcutaneously). Calcium image sequences were acquired at depths of up to 100 μm at 1-3 Hz at intervals of 4-6 seconds.

Laminin α4 Knockout Mouse Model. Subcutaneous fat was separately collected from laminin α4 knock out mice and wild-type mice. The samples were processed and incubated with integrin a7 antibody (1:100, Novus Biologics NBP1-86118) and Griffonia simplicifolia isolectin conjugated with Rhodamine (labels endothelial cells/blood vessels) followed by incubation with a second antibody (Alexa Fluor 647 Donkey Anti-Rabbit IgG, Abcam ab150075) and BODIPY to stain lipid. Images were collected by a Leica TCS SP8 Confocal Microscope.

NPC calcium image acquisition and processing. ReNCell VM neural progenitor cells were plated on LabTek chambered cover glasses for calcium imaging experiments. Cells were loaded with culture medium containing 3 μM of the fluorescent calcium indicator Fluo-4/AM (Life Technologies) and Pluronic F-127 (0.2% w/v, Life Technologies) for 30 min at 37° C. Imaging of spontaneous calcium activity was performed at 37° C. using a 20× objective lens (N.A.=0.75), with 488 nm excitation provided through a SOLA SE Light Engine (Lumencor). 16-bit fluorescence images were acquired at a sampling frequency of 1 Hz for a total duration of 15 min, using a Zyla 5.5 sCMOS camera (Andor). Following calcium imaging, samples were fixed, and nuclei were stained using DAPI. By navigating to the locations where calcium imaging was performed, manual co-registration was done to obtain immunofluorescence images for the same fields of view.

Regions of interest (ROIs) were obtained by segmenting nucleus images using a local thresholding approach followed by the watershed algorithm. Undersegmented objects were algorithmically removed by discarding the top two percentile of object sizes obtained after segmentation. Next, a time-varying fluorescence trace was calculated for each ROI. For each frame in the calcium fluorescence image stack, background (average pixel intensity of non-ROI regions in the image) was subtracted. Average fluorescence intensity for each ROI (F) was obtained by averaging pixel intensity values within the ROI for each time point. Baseline fluorescence (F₀) for each ROI was calculated as the minimum intensity value in a window 90 s before and after each time point. The normalized fluorescence trace for the ROI was then calculated as F−F₀/F₀. Cells with low activity were filtered out by discarding traces with less than three peaks and traces whose signal to-noise ratio was lower than 1. Quality of the remaining traces was confirmed by manual inspection. This was done to avoid false positives in the cross-correlation analysis.

Generation of FUCCI Reporter Neural Progenitor Cell Lines. Stable reporter cell lines (FUCCI-ReN) were generated by sequentially nucleofecting ReNcell VM neural progenitor cells with an ePiggyBac (146) construct encoding mCherry-Cdt, Venus-Geminin, or Cerulean-H2B. Each construct introduced to the cells was driven by a CAG promoter containing a blasticidin (ePB-B-CAG-mCherry-Cdt1), puromycin (ePB-P-Venus-Geminin), or neomycin (ePB-N-Cerulean-H2B) resistance gene. Following each round of nucleofection, cells were cultured in the presence of appropriate antibiotics (2 μg/ml blasticidin, 0.1 μg/ml puromycin and 100 μg/ml neomycin).

Acquisition and processing of FUCCI-ReN time lapse videos. FUCCI-ReN cells were plated at different densities on chambered cover glasses (Fisher Scientific) coated with laminin. Cells were imaged after switching to differentiation medium containing phenol red-free DMEM/F12. Time-lapse imaging was performed using a Nikon Ti-E microscope equipped with a motorized stage, a cage incubator for environmental control (Okolab), a 20× objective lens (N.A.=0.75), SOLA SE Light Engine for LED-based fluorescence excitation (Lumencor), appropriate filters for visualizing mCherry, Venus and Cerulean fluorescent proteins and a Zyla 5.5 sCMOS camera (ANDOR). 16-bit composite fluorescence images were acquired at 10-minute intervals for a total duration of 57.5 hours.

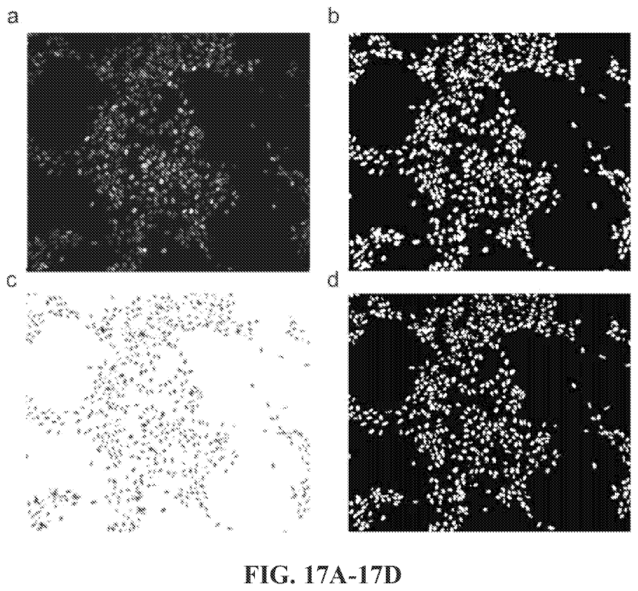

Grayscale images for each channel (H2B-Cerulean, Geminin-Venus and Cdt1-mCherry) were binarized using locally adaptive thresholding. Seeds for the watershed transform were generated using the regional minima from the distance transform of the grayscale images. Next, the watershed algorithm was applied to detect boundaries between overlapping cell nuclei. Finally, information from different channels were used to correct undersegmented nuclei (FIG. 17).

Acquisition and processing of HUVEC immunocytochemistry images. For imaging experiments, HUVECs were cultured on glass dishes coated with fibronectin (Sigma-Aldrich). After appropriate growth factor treatments, cultures were fixed with 4% paraformaldehyde, free aldehyde groups were quenched using 1 mg/mL sodium borohydride, and membranes were permeabilized with 0.2% Triton-X-100 solution in PBS. Actin fibers were visualized using an Alexa Fluor 488-phalloidin antibody (1:40, Molecular Probes) and microtubules were visualized using a mouse monoclonal anti-α-Tubulin antibody (1:250, Sigma-Aldrich) followed by a goat anti-mouse Alexa Fluor 647 secondary antibody. Nuclei were stained using Hoescht (Molecular Probes). 16-bit composite immunofluorescence images were acquired through a 20× objective (N.A.=0.75) on a Nikon Ti-E epifluorescence microscope. Physical pixel size was 0.32 μm.

Fluorescence images were processed as described previously (147) (FIG. 16).

Briefly, the following steps were used: 1. Contrast was enhanced using histogram equalization. 2. Images were smoothed using a 2D Gaussian lowpass filter. 3. Initial binarization was performed using Otsu's method. 4. The binary image was dilated to fill in individual cell areas. 5. All objects <1% of the total image area were removed. This was called the final binary image. 6. A binary representation of the nuclear and microtubule image layers was generated using a high input threshold value. This was called the marker image. 7. Another binary image was created with values of 0 where either the final binary image (step 5) or the marker image (step 6) had a value of 1.8. Watershed markers were generated by imposing the minimum of the complement of images obtained in steps 2 and 7. This image had black markers contained within cells to serve as basins for flooding, while cell areas themselves were represented by lighter pixels that served as the rising contours of the basins. 9. The watershed algorithm was implemented using Matlab's built-in function to generate cell boundaries. 10. Masks generated in step 9 were refined by using composite images of microtubules and actin as the marker image (step 6).

In order to automate the threshold generation, the area of cell masks obtained from segmentation were compared to those obtained through thresholding with a high threshold. The entire process was then iterated until an acceptable area ratio was achieved.

Processing of In Vivo Calcium Image Sequences. Calcium image sequences from dorsal root ganglion models were processed as follows. To generate a mask, the calcium image sequence was first decomposed into individual grayscale frames. Next, for each pixel location, the maximum and minimum intensities were found across all frames. The differences between the maximum and minimum intensities were stored in an array (of delta values) and normalized. An initial segmentation of the delta values was done by thresholding using Otsu's method, resulting in an initial binary mask. The initial mask was refined by computing a new threshold by applying Otsu's method to only those delta values that were identified as foreground objects in the initial segmentation. The resulting binary image underwent a morphological closing with a disk of radius 3, and objects of fewer than 10 pixels were removed to generate the final mask.

To generate functional networks, edges were placed between two cells whenever: (a) the two cells had the same ramp-up and ramp-down times, and (b) the Euclidean distance between the centroids of the two cells was less than or equal to 10 times the mean of the diameter of each of the two cells.

Cluster Analysis. Cluster analysis was performed on the HUVEC imaging dataset using Shrinkage Clustering (39), a two-in-one clustering and cluster optimization algorithm based on matrix factorization that simultaneously finds the optimal number of clusters while partitioning the data. Cells whose features had the smallest sum of squares distance to the median values for each cluster were identified as representative cells for each cluster.

Correction of Morphology Metrics for Effects of Local Network Properties and Treatment Conditions. Quantile multidimensional binning (149) of cells was performed for all 7 network metrics (5 bins 613 per metric). The mean of each morphology metric was calculated for each multidimensional bin, and this mean was subtracted from the raw measurements to generate the network-corrected measurements for each cell. Treatment-corrected measurements were generated similarly by calculating the mean of each morphology metric under each treatment condition and then subtracting it from the raw measurements.

Variance Explained by Local Network Properties and Treatment Conditions. The variance explained by each factor was calculated using the following formula (137)

$$1 - {V}_{corr} / {V}_{uncorr}$$

V_{corr} is the variance of the corrected measurements, and V_{uncorr} is the variance of the uncorrected measurements.

### REFERENCES

1. Mahadevan et al., bioRxiv 055533v3(2021).
2. Long et al., J Neurosci Methods 283, 62-71 (2017).
3. Francis et al., Nat Med 22, 388-396 (2016).
4. Mahadevan et al., PLOS Computational Biology in review (2021).
5. Mahadevan et al., URL braininitiative.org/toolmakers/resources/cytonet/, 2021.
6. Yang et al., Lab Chip 21, 435-446 (2021).
7. Yang et al., Bioengineering (Basel) 7(2020).
8. Britton et al., Development 146(2019).
9. Irwin, Annual Review of Psychology 66, 143-172 (2015).
10. Sigl-Glöckner and Seibt, Journal of Neuroscience Methods, Vol. 316 71-82 (Elsevier, 2019).
11. Chan Kwong et al., J Clin Psychopharmacol 40, 222-230 (2020).
12. Besedovsky et al., Physiological Reviews 99, 1325-1380 (2019).
13. Shokri-Kojori et al., Proc Natl Acad Sci USA 115, 4483-4488 (2018).
14. Spira et al., JAMA Neurol 70, 1537-1543 (2013).
15. Fultz et al., Science 366, 628-631 (2019).
16. Shrestha et al., Tissue Eng Part B Rev 26, 79-102 (2020).
17. Appel et al., Tissue Eng Part C Methods 22, 1038-1048 (2016).
18. Sokic et al., Microvasc Res 92, 72-78 (2014).
19. Roux et al., Tissue Eng Part A (2020).
20. Acosta et al., Tissue Eng Part A 26, 905-914 (2020).
21. Langert and Brey, Front Neurosci 12, 887 (2018).
22. Sanchez et al., J Tissue Eng Regen Med 12, 2164-2178 (2018).
23. Roux et al., Tissue Eng Part A 24, 1603-1615 (2018).
24. Pollet and Qutub, Survey on activities supporting cognitive health during the COVID-19 pandemic (2020).
25. Pollet et al., medRxiv 2020.12.07.20245001(2020).
26. Long et al., J Theor Biol 326, 43-57 (2013).
27. Ozdemir-Kaynak et al., Frontiers in Physiology, Vol. 9 (2018).
28. Qutub and Hunt, Brain Research Reviews, Vol. 49 (2005).
29. Tang et al., Sci Rep 9, 12529 (2019).
30. Nathan and Qutub, Patient-Specific Modeling of the Cardiovascular System: Technology-Driven Personalized Medicine (2010).
31. Noren et al., Sci Signal 9, ra20 (2016).
32. Qutub et al., Multiscale Modeling of Particle Interactions: Applications in Biology and Nanotechnology (2010).
33. Qutub et al., Pac Symp Biocomput, 316-327 (2009).
34. Qutub et al., IEEE Eng Med Biol Mag 28, 14-31 (2009).
35. Qutub and Popel, Journal of Cell Science, Vol. 119 (2006).
36. Qutub and Popel, BMC Systems Biology, Vol. 3 (2009).
37. Qutub and Popel, Molecular and Cellular Biology, Vol. 28 (2008).
38. Hu et al., Sci Rep 5, 12894 (2015).
39. Hu et al., BMC Bioinformatics 19, 19 (2018).
40. Hu et al., Nat Biomed Eng 3, 889-901 (2019).
41. Slater et al., ACS Nano. 2015; 9(6):6128-38.
42. Alon et al., Science 371(2021).
43. Otsuki and Brand, Science 360, 99-102 (2018).
44. Gil-Ranedo et al., Cell Rep 27, 2921-2933 e2925 (2019).
45. Friedmann-Morvinski et al., Science 338, 1080-1084 (2012).
46. Li and Chen, Neuron 91, 728-738 (2016).
47. Lu et al., Nature 588, 124-129 (2020).
48. Choudhury et al., Glia 68, 44-59 (2020).
49. Becquet et al., Glia 56, 294-305 (2008).
50. Zada et al., Nature Communications, Vol. 10 (Springer US, 2019).
51. Tabuchi et al., Cell 175, 1213-1227 e1218 (2018).
52. Macknik et al. J Neurosci 39, 8267-8274 (2019).
53. Djonlagic et al., Nat Hum Behav 5, 123-145 (2021).
54. Hirten et al., J Med Internet Res 23, e26107 (2021).
55. Refinetti, Yale J Biol Med 92, 179-186 (2019).
56. Morelli et al., Physiol Meas 40, 095001 (2019).
57. Vaidyanathan et al., Elife 10(2021).
58. Pollet et al., Sleep 43, A640-A461 (2020).
59. Penninckx et al., Radiat Res 195, 47-59 (2021).
60. Georgescu et al., PLOS One 10, e0129438 (2015).
61. Jiang et al., J Neurol Neurosurg Psychiatry (2020).
62. Abdel-Haq, Neural Regeneration Research, Vol. 14 72-74 (2019).
63. Sharma et al., PNAS United States of America, Vol. 116 16086-16094 (2019).
64. Vella et al., Journal of Extracellular Vesicles, Vol. 6 (Taylor & Francis, 2017).
65. Fiandaca et al., Alzheimers Dement 11, 600-607 e601 (2015).
66. Anastasi et al., Int J Mol Sci 22(2021).
67. Ignjatovic et al., J Proteome Res 18, 4085-4097 (2019).
68. Hu et al., Scientific Reports, Vol. 5 (2015).
69. De Luca Canto et al., Sleep Med 16, 347-357 (2015).
70. Julian et al., J Strength Cond Res 31, 608-619 (2017).
71. Okun et al., Brain Behav Immun 23, 351-354 (2009).
72. Cheung et al., Anaesthesia 74, 434-440 (2019).
73. Horowitz et al., Science 369, 167-173 (2020).
74. Villeda et al., Nature 477, 90-94 (2011).
75. Fernandez et al., PNAS USA 113, 6047-6052 (2016).
76. Achberger et al., Adv Drug Deliv Rev 140, 33-50 (2019).
77. Nakano et al., Cell Stem Cell 10, 771-785 (2012).
78. Eiraku et al., Nature 472, 51-56 (2011).
79. Hayashi et al., Nature 531, 376-380 (2016).
80. Kano et al., J Endocr Soc 5, bvaa188 (2021).
81. Mitsumoto et al., Stem Cell Research, Vol. 40 101572 (Elsevier, 2019).
82. Kim et al., Nat Commun 11, 4360 (2020).
83. Ogawa et al., Scientific Reports, Vol. 8 1-10 (Springer US, 2018).
84. Bedont et al., Wiley Interdiscip Rev Dev Biol 4, 445-468 (2015).
85. Landgral et al., Frontiers in Neuroanatomy, Vol. 8 1-7 (2014).
86. Patel et al., Journal of Neuroscience Methods, Vol. 243 26-38 (Elsevier B.V., 2015).
87. Herzog et al., Methods Enzymol 552, 3-22 (2015).
88. Muter et al., FASEB J 29, 1603-1614 (2015).
89. Nunez et al., Cell 184(9): P2503-2519.e17, Apr. 29, 2021.
90. Shi et al., PLOS Biol 18, e3000705 (2020).
91. Manafi et al., Ocul Surf 19, 1-15 (2021).
92. Shiju et al., Exp Eye Res 200, 108213 (2020).
93. Foster et al., Methods Mol Biol 2145, 51-58 (2020).
94. Susaimanickam et al., Development 144, 2338-2351 (2017).
95. Guo et al., Front Cell Neurosci 13, 361 (2019).
96. Boutin et al., Adv Exp Med Biol 1186, 171-193 (2019).
97. Mei et al., PNASci USA 115, 4276-4281 (2018).
98. Liang et al., Nips'15, 937-945 (2015).
99. Noren et al., PLOS Computational Biology, Vol. 12 (2016).
100. Hill et al., Nat Methods 13, 310-318 (2016).
101. Qutub, Brain Health Workshops: URL www.quantuproject.org/workshops.
102. Qutub, Biomedical Data Workshops: URL www.qutublab.org/when.
103. Qutub, NSF NCS Neuro Lab Workshops: URL www.qutublab.org/neuro.
104. Hu et al., R Journal, Vol. 8 (2016).
105. Gonzalez-Porras et al., Scientific Reports 2021 11, Article number: 5442 2021.
106. Meijering et al., Nature Biotechnology 2016 34(12): 1250-5.
107. Mund et al., bioRxiv 2021 2021.01.25.427969.
108. Bray et al., Nature Protocols 2016 11(9): 1757-74.
109. McQuin et al., PLOS Biology 2018 1-17.
110. Kuthuru et al., Cancer Inform 2019 18:1176935119856595.
111. Schürch et al., Cell 2020 182(5): 1341-59.e19.
112. Vickovic et al., Nature Methods 2019 16(10): 987-90.
113. Gut et al., Science 2018 361(6401).
114. Eng et al., Nature 2019 568(7751): 235-9.
115. Thul et al., Science 2017 356(6340).
116. Bassett et al., Nature Reviews Neuroscience 2018 19(9):566-78.
117. Bassett and Sporns, Nature Neuroscience 2017 20(3): 353-64.
118. Yener and Bülent, Communications of the ACM 2016 60(1): 74-84.
119. Qutub et al. Automated method for measuring, classifying, and matching the dynamics and information passing of single objects within one or more images 2013.
120. Rekhi et al. In: Zhang G, editor. Computational Bioengineering: CRC Press; 2015. p. 254-82.
121. Blankenship and Feller, Nature reviews Neuroscience 2010 11(1): 18-29.
122. Malmersjö et al., PNAS USA 2013 110:E1524-32.
123. Shimojo et al., Neuron 2008 58:52-64.
124. Otani et al. Cell Stem Cell 2016 18(4):467-80.
125. Li et al., Cell Stem Cell 2016 19(1):120-6.
126. Cai et al., The Journal of neuroscience 1997 17(6):2088-100.
127. Reznikov et al., The Journal of comparative neurology 1995 360(3):536-54.
128. Sakaue-Sawano et al., Cell 2008 132(3):487-98.
129. Megat et al., J Neurosci 2019 39(35): 6829-47.
130. Kim et al., Neuron 2016 91(5): 1085-96.
131. Kim et al., Neuron 2014 81(4):873-87.
132. López-Cancio et al., Journal of Stroke and Cerebrovascular Diseases 2017 26(2):425-30.
133. Wei et al., Journal of Cerebral Blood Flow & Metabolism 2017 0271678X1770266-0271678X1770266.
134. Goodwin, Microvascular Research 2007 74(2): 172-83.
135. Costa et al., Nature Cell Biology. 2016 18(12): 1292-301.
136. Snijder et al., Nature 2009 461(7263):520-3.
137. Gut et al., Nature methods 2015 12(10):951-4.
138. Vaicik et al., PLOS One 2014 9(10): e109854.
139. Vaicik et al., Endocrinology 2018 159(1):356-67.
140. Vaicik et al., J Mater Chem B 2015 3(40): 7903-11.
141. Berg et al., Nature Methods. 2019 16(12): 1226-32.
142. Hollandi et al., Cell Systems 2020 10(5):453-8.e6.
143. Moen et al., bioRxiv. 2019 803205.
144. Carpenter et al., Genome Biology 2006 7(10):R100-R.
145. Smedler et al., Front Neural Circuits 2014 8:111.
146. Bounova et al., Physical Review E—Statistical, Nonlinear, and Soft Matter Physics 2012 85.
147. Lacoste et al., Cell Stem Cell 2009 5(3):332-42.
148. Ryan et al., DOI: 10.1115/NEMB2013-93124.
149. Snijder et al., Molecular Systems Biology 2012: 8.

1. A method of evaluating a living system comprising:
(a) measuring in vivo physiologic, behavioral, or physiologic and behavioral characteristics of a living subject to obtain non-invasive data;
(b) establishing an in vitro cell model of a cellular network, exposing the in vitro cell model to a condition(s) to model a cellular environment in the living subject, and measuring cellular changes to obtain in vitro model data;
(c) transforming the non-invasive data to functional graphs;
(d) transforming the in vitro model data to topological, functional, or topological and functional graphs; and
(e) integrating the non-invasive graphs and the in vitro model graph using a neural network.

2. The method of claim 1, wherein transforming the non-invasive data to topological and functional graphs utilizes cytoNet software.

3. The method of claim 1, wherein transforming the in vitro model data to topological and functional graphs utilizes cytoNet software.

4. The method of claim 1, wherein the non-invasive data comprises one or more of non-invasive imaging, biomarker analysis, or bio-electrical patterns.

5. The method of claim 4, wherein bio-electrical patterns comprise electroencephalograms.

6. The method of claim 4, wherein non-invasive imaging comprises retinal scans.

7. The method of claim 4, wherein non-invasive imaging comprises biomarker analysis of a blood sample.

8. The method of claim 1, wherein the neural network is a long short-term memory network (CNN-LSTM).

9. A method for defining a sleep signature for a subject comprising:
(a) plotting frequencies of sleep stages of a subject over a period of time, wherein the sleep stages are light sleep (L), deep sleep (D), rapid eye movement (REM) sleep, and wake (W);
(b) identifying sleep stage motifs in the sleep stage plot.

10. The method of claim 9, wherein sleep stages are determined using a wearable device.

11. The method of claim 9, wherein sleep stage motifs are identified by scanning a window of sleep stage sequence by comparing a scan window to a position frequency matrix and assessing a probability of the sequence using a position probability matrix and determining the probability of the motif.

12. A method of identifying sleep motifs comprising: (i) obtaining electroencephalogram (EEG) data, (ii) measuring and analyzing heartrate data to characterize deviations in circadian rhythm, and (iii) analyzing EEG and heart rate data to identify motifs in the data characteristic of a sleep signature.

13. A method for identifying sleep signatures predictive of cognitive performance change in response to exercise comprising, using data from sensor devices or wearables comprising:
(i) obtaining electroencephalogram (EEG) of a plurality of sleeping subjects;
(ii) measuring and analyzing heartrate data to characterize deviations in circadian rhythm in the plurality of subjects;
(iii) classify the subjects into sub-groups defined sleep quality; and
(iv) analyzing sleep data to find motifs in the data by comparing the subjects in a sub-group and identifying the common sleep signature within the sub-group.

14. A method of identifying molecular (epigenetic) or cellular (neuronal) biomarkers of sleep quality comprising:
(i) obtaining a biological sample from a plurality of subjects exhibiting a particular sleep signature;
(ii) identifying one or more molecular (epigenetic) or cellular (neuronal) biomarker that correlates with the particular sleep signature.

15. An experimental, human cell-based brain model of regions of the brain regulating sleep, mood and circadian rhythmicity comprising in vitro differentiated inducible pluripotent stem cells (iPSCs) that are differentiated to hypothalamic cells by activation of transcription factors Six3, Six6, Lhx2, and Lhx1 forming the hypothalamic cells, the hypothalamic cells are determined to exhibit functional connectivity associated with the suprachiasmatic nucleus (SCN).
